(19) United States
(12) Patent Application Publication
(10) Pub. No.: US 2017/0216227 A1
(43) Pub. Date: Aug. 3, 2017

(54) DELIVERY MOLECULES FOR THERAPEUTICS

(71) Applicant: Marina Biotech, Inc., City of Industry, CA (US)

(72) Inventors: Renata Fam, Kenmore, WA (US); Roger C. Adami, Snohomish, WA (US); Kathy L. Fosnaugh, Woodinville, WA (US); Pierrot Harvie, Bothell, WA (US); Rachel E. Johns, Shoreline, WA (US); Shaguna Seth, Bothell, WA (US); Michael E. Houston, JR., Sammamish, WA (US); Michael V. Templin, Bothell, WA (US)

(21) Appl. No.: 15/487,374

(22) Filed: Apr. 13, 2017

Related U.S. Application Data
(63) Continuation of application No. 14/542,653, filed on Nov. 16, 2014, now abandoned.
Application No. 14/542,653 is a continuation of application No. 13/505,685, filed on Jul. 18, 2012, now abandoned.
(60) Provisional application No. 61/258,115, filed on Nov. 4, 2009.

(51) Int. Cl. A61K 31/16; A61K 9/127; C07C 237/52; C07D 233/64; C12N 15/88; C12N 15/113; A61K 31/4172; A61K 31/401; A61K 31/713; A61K 9/00; C07D 207/16

(52) U.S. Cl. CPC A61K 31/16; A61K 9/0019; A61K 9/127; C07C 237/52; C07D 233/64; C07D 207/16; C12N 15/1137; A61K 31/4172; A61K 31/401; A61K 31/713; C12N 15/88; C12N 2310/14; C12N 2320/32

(57) ABSTRACT

Activity-generating delivery molecules comprising the structure R³—(C═O)-Xaa-NH—R⁴ wherein Xaa is any D- or L-amino acid residue with a non-hydrogen, substituted or unsubstituted side chain, R³—(C═O)— and —NH—R⁴ are independently a long chain group, each long chain group containing one or more carbon-carbon double bonds, and salts, compositions and methods of use thereof. The activity-generating delivery compounds and compositions are useful for generating activity of an active agent in a cell, tissue, or subject.

### TECHNICAL FIELD

This invention relates generally to molecules, compositions, methods and uses for generating activity of biologically active agents and therapeutic agents by delivering the agents to selected cells, tissues, and organs, as well as to subjects. More particularly, embodiments of this invention include molecules and compositions useful for delivery of therapeutic agents including nucleic acid agents, and methods and uses for effecting drug delivery and generating biological activity.

### BACKGROUND

Biomolecules and biopharmaceutical molecules designed to be biologically or pharmacologically active for a selected target have an activity that can be established in an assay. The assay is used to search for, among other things, the most active molecules with respect to the chosen target. Once the active molecules or moieties are identified, the goal is to develop a drug for administration to a subject that can reach the desired target and induce drug effects.

Some biologically active molecules are susceptible to attack and degradation through a variety of mechanisms upon administration to a subject. The delivery of a therapeutic molecule can be impeded by limited ability of the compound to reach a target cell or tissue, or by restricted entry through membranes or trafficking of the compound within cells.

The use of a biologically active molecule as a drug may therefore depend entirely on the ability to transport and deliver it to the interior of cells. One strategy to deliver an active molecule is to combine or pair it with a synthetic carrier molecule. The carrier molecule can provide the transport and delivery properties which generate the biological activity in a cell, tissue or other target. This means that the search for a therapeutic system can essentially become the search for an effective synthetic carrier molecule.

A carrier molecule can protect an active agent from degradation, for example, by encapsulating or binding to the active agent. In addition, a carrier molecule can greatly increase uptake in cells of an active agent by interacting with negatively charged cell membranes to initiate transport across a membrane.

For example, recent advances have increased the need for effective means of introducing active nucleic acid agents into cells. Nucleic acid agents such as gene-silencing agents, gene-regulating agents, RNA interference agents, antisense agents, as well as peptide nucleic acid agents, ribozyme agents, RNA agents, and DNA agents in general may advantageously be delivered with carrier molecules.

What is needed are processes, compositions, and uses for systemic and local delivery of drugs and biologically active molecules including nucleic acid agents. Among other things, there is a longstanding need for delivery compositions, structures and carriers that can increase the efficiency of delivery of biologically active and therapeutic molecules.

### BRIEF SUMMARY

This disclosure provides novel processes, compositions and formulations for intracellular and in vivo delivery of drug agents for use, ultimately, as a therapeutic, that in general maintain cytoprotection and relatively low toxicity. The methods and compositions of this disclosure are useful for delivery of drug agents to selected cells, tissues, and organs.

In some aspects, this disclosure provides processes, compositions and methods to deliver active nucleic acid agents or molecules to cells. The active agents may provide therapeutic or pharmacological effects, either through pharmaceutical action, or by producing the response of RNA interference, or antisense or ribozyme effects. Active agents of this disclosure may be useful in the regulation of genomic expression, or for gene therapy.

Embodiments of this invention include activity-generating delivery molecules comprising an amino acid having a long chain alkenoyl group at the N-terminus and a long chain alkenylamino group at the C-terminus, wherein each long chain group has from 12 to 24 carbon atoms and one or more carbon-carbon double bonds.

In some embodiments, an activity-generating delivery molecule may have at least one long chain group with two or more carbon-carbon double bonds.

Embodiments of this invention include compounds comprising the structure shown in Formula I: R³—(C═O)-Xaa-NH—R⁴ (Formula I) wherein Xaa is any D- or L-amino acid residue having the general formula —NR^{N}—CR¹R²—(C═O)—, wherein R¹ is a non-hydrogen, substituted or unsubstituted side chain of an amino acid;
R², R^{N} are independently hydrogen, or an organic group consisting of carbon, oxygen, nitrogen, sulfur, and hydrogen atoms, and having from 1 to 20 carbon atoms, or C(1-5)alkyl, cycloalkyl, cycloalkylalkyl, C(3-5)alkenyl, C(3-5)alkynyl, C(1-5)alkanoyl, C(1-5)alkanoyloxy, C(1-5)alkoxy, C(1-5)alkoxy-C(1-5)alkyl, C(1-5)alkoxy-C(1-5)alkoxy, C(1-5)alkyl-amino-C(1-5)alkyl-, C(1-5)dialkyl-amino-C(1-5)alkyl-, nitro-C(1-5)alkyl, cyano-C(1-5)alkyl, aryl-C(1-5)alkyl, 4-biphenyl-C(1-5)alkyl, carboxyl, or hydroxyl;
R³—(C═O)— is independently a long chain group which may be derived from a naturally-occurring phospholipid, glycolipid, triacylglycerol, glycerophospholipid, sphingolipid, ceramide, sphingomyelin, cerebroside, or ganglioside, wherein the long chain group contains one or more carbon-carbon double bonds; or a substituted or unsubstituted C(12-24)alkenoyl;
—NH—R⁴ is independently a long chain group which may be derived from a naturally-occurring phospholipid, glycolipid, triacylglycerol, glycerophospholipid, sphingolipid, ceramide, sphingomyelin, cerebroside, or ganglioside, wherein the long chain group contains one or more carbon-carbon double bonds; or a
substituted or unsubstituted C(12-24)alkenylamino;
   and salts thereof.

An activity-generating delivery molecule may have R³—(C═O)— is independently a substituted or unsubstituted C(12-24)alkenoyl and —NH—R⁴ is independently a substituted or unsubstituted C(12-24)alkenylamino.

An activity-generating delivery molecule may have R³,R⁴ are each independently C12 alkenyl, C13 alkenyl, C14alkenyl, C15alkenyl, C16alkenyl, C17alkenyl, C18alkenyl, C19alkenyl, C20alkenyl, C21alkenyl, C22alkenyl, C23alkenyl, or C24alkenyl.

An activity-generating delivery molecule may have:

R³—(C═O)— is independently C12alkenoyl, C13alkenoyl, C14alkenoyl, C15alkenoyl, C16alkenoyl, C17alkenoyl, C18alkenoyl, C19alkenoyl, C20alkenoyl, C21alkenoyl, C22alkenoyl, C23alkenoyl, or C24alkenoyl; and —NH—R⁴ is independently C12alkenylamino, C13alkenylamino, C14alkenylamino, C15alkenylamino, C16alkenylamino, C17alkenylamino, C18alkenylamino, C19alkenylamino, C20alkenylamino, C21alkenylamino, C22 alkenylamino, C23alkenylamino, or C24alkenylamino.

An activity-generating delivery molecule may have:

R³—(C═O)— is independently C(12:1)alkenoyl, C(12:2)alkenoyl, C(12:3)alkenoyl, C(14:1)alkenoyl, C(14:2)alkenoyl, C(14:3)alkenoyl, C(16:1)alkenoyl, C(16:2)alkenoyl, C(16:3)alkenoyl, C(18:1)alkenoyl, C(18:2)alkenoyl, C(18:3)alkenoyl, C(18:4)alkenoyl, C(20:1)alkenoyl, C(20:2)alkenoyl, C(20:3)alkenoyl, C(20:4)alkenoyl, C(20:5)alkenoyl, C(22:1)alkenoyl, C(22:4)alkenoyl, or C(22:6)alkenoyl; and

—NH—R⁴ is independently C(12:1)alkenylamino, C(12:2)alkenylamino, C(12:3)alkenylamino, C(14:1)alkenylamino, C(14:2)alkenylamino, C(14:3)alkenylamino, C(16:1)alkenylamino, C(16:2)alkenylamino, C(16:3)alkenylamino, C(18:1)alkenylamino, C(18:2)alkenylamino, C(18:3)alkenylamino, C(18:4)alkenylamino, C(20:1)alkenylamino, C(20:2)alkenylamino, C(20:3)alkenylamino, C(20:4)alkenylamino, C(20:5)alkenylamino, C(22:1)alkenylamino, C(22:4)alkenylamino, or C(22:6)alkenylamino.

An activity-generating delivery molecule may have:

R³—(C═O)— is independently C(14:1(5))alkenoyl, C(14:1(9))alkenoyl, C(16:1(7))alkenoyl, C(16:1(9))alkenoyl, C(18:1(3))alkenoyl, C(18:1(5))alkenoyl, C(18:1(7))alkenoyl, C(18:1(9))alkenoyl, C(18:1(11))alkenoyl, C(18:1(12))alkenoyl, C(18:2(9,12))alkenoyl, C(18:2(9,11))alkenoyl, C(18:3(9,12,15))alkenoyl, C(18:3(6,9,12))alkenoyl, C(18:3(9,11,13))alkenoyl, C(18:4(6,9,12,15))alkenoyl, C(18:4(9,11,13,15))alkenoyl, C(20:1(9))alkenoyl, C(20:1(11))alkenoyl, C(20:2(8,11))alkenoyl, C(20:2(5,8))alkenoyl, C(20:2(11,14))alkenoyl, C(20:3 (5,8,11))alkenoyl, C(20:4(5,8,11,14))alkenoyl, C(20:4(7,10,13,16))alkenoyl, C(20:5(5,8,11,14,17))alkenoyl, C(20:6(4,7,10,13,16,19))alkenoyl, C(22:1(9))alkenoyl, C(22:1(13))alkenoyl, or C(24:1(9))alkenoyl; and

—NH—R⁴ is independently C(14:1(5))alkenylamino, C(14:1(9))alkenylamino, C(16:1(7))alkenylamino, C(16:1(9))alkenylamino, C(18:1(3))alkenylamino, C(18:1(5))alkenylamino, C(18:1(7))alkenylamino, C(18:1(9))alkenylamino, C(18:1(11))alkenylamino, C(18:1(12))alkenylamino, C(18:2(9,12))alkenylamino, C(18:2(9,11))alkenylamino, C(18:3(9,12,15))alkenylamino, C(18:3(6,9,12))alkenylamino, C(18:3(9,11,13))alkenylamino, C(18:4(6,9,12,15))alkenylamino, C(18:4(9,11,13,15))alkenylamino, C(20:1(9))alkenylamino, C(20:1(11))alkenylamino, C(20:2(8,11))alkenylamino, C(20:2(5,8))alkenylamino, C(20:2(11,14))alkenylamino, C(20:3(5,8,11))alkenylamino, C(20:4(5,8,11,14))alkenylamino, C(20:4(7,10,13,16))alkenylamino, C(20:5(5,8,11,14,17))alkenylamino, C(20:6(4,7,10,13,16,19))alkenylamino, C(22:1(9))alkenylamino, C(22:1(13))alkenylamino, or C(24:1(9))alkenylamino.

In some embodiments, this invention provides compositions comprising an activity-generating delivery molecule contacted with an active agent.

In some embodiments, this invention provides compositions comprising an activity-generating delivery molecule contacted with an active nucleic acid agent.

In some embodiments, this invention provides compositions comprising an activity-generating delivery molecule contacted with an active RNA agent.

In some embodiments, this invention provides compositions comprising an activity-generating delivery molecule contacted with a UsiRNA agent.

In some embodiments, this invention provides compositions comprising an activity-generating delivery molecule contacted with a siRNA agent.

In some embodiments, this invention provides compositions comprising an activity-generating delivery molecule admixed with a lipid, a cationic lipid, or a non-cationic lipid.

This invention may further provide methods for delivering a therapeutic nucleic acid to a cell comprising contacting the cell with a formulation containing an activity-generating delivery molecule and a nucleic acid agent.

In certain aspects, this invention includes methods for inhibiting expression of a gene in a cell comprising contacting the cell with a formulation containing an activity-generating delivery molecule and a nucleic acid agent.

In further aspects, this invention includes methods for inhibiting expression of a gene in a mammal comprising administering to the mammal a formulation containing an activity-generating delivery molecule and a nucleic acid agent.

In some embodiments, this disclosure includes methods for treating a disease in a human comprising administering a formulation containing an activity-generating delivery molecule and a nucleic acid agent to the human, wherein the disease is cancer, bladder cancer, cervical cancer, liver cancer, liver disease, hypercholesterolemia, an inflammatory disease, a metabolic disease, inflammation, arthritis, rheumatoid arthritis, encephalitis, bone fracture, heart disease, and viral disease.

In certain embodiments, an activity-generating delivery molecule may be used in treating a disease in a human including cancer, bladder cancer, cervical cancer, liver cancer, liver disease, hypercholesterolemia, an inflammatory disease, a metabolic disease, inflammation, arthritis, rheumatoid arthritis, encephalitis, bone fracture, heart disease, and viral disease.

This invention includes uses of a formulation containing an activity-generating delivery molecule and a nucleic acid agent for treating a disease including cancer, bladder cancer, cervical cancer, liver cancer, liver disease, hypercholesterolemia, an inflammatory disease, a metabolic disease, inflammation, arthritis, rheumatoid arthritis, encephalitis, bone fracture, heart disease, and viral disease.

This invention includes uses of a formulation containing an activity-generating delivery molecule and a nucleic acid agent in the preparation of a medicament for treating a disease including cancer, bladder cancer, cervical cancer, liver cancer, liver disease, hypercholesterolemia, an inflammatory disease, a metabolic disease, inflammation, arthritis, rheumatoid arthritis, encephalitis, bone fracture, heart disease, and viral disease.

Additional features and benefits of this invention are apparent from the detailed description below, as well as from the attached drawings and claims, which taken together as a whole encompass the disclosure of this invention.

### BRIEF DESCRIPTION OF THE DRAWINGS

FIG. 1: In FIG. 1 is shown a chart of the gene-silencing dose-response in vivo mouse for a UsiRNA against Factor V-II administered by tail-vein injection in a formulation including an activity-generating delivery molecule of this invention C18:2-DAP(N,N-diMe)-C18:2. The calculated ED50 was 30 μg/kg.

FIG. 2: In FIG. 2 is shown a chart of the 2nd melting behavior of the compound CH₃(CH₂)₁₆(CO)-norArg-NH(CH₂)₁₇CH₃ assessed by differential scanning calorimetry. The large peaks indicate the presence of significant thermal or melting transitions.

FIG. 3: In FIG. 3 is shown a chart of the 2nd melting behavior of the compound C(18:2)oleoyl-DAB-C(18:2)alkenylamino assessed by differential scanning calorimetry, which represents an embodiment of this invention. The DSC scan in FIG. 3 reveals the complete lack of thermal transition peaks in the DSC.

### DETAILED DESCRIPTION

This disclosure provides a range of compounds, compositions, formulations, and uses directed ultimately toward drug delivery, including therapeutics and the diagnosis and treatment of diseases and conditions.

In some embodiments, this invention provides a range of compounds, compositions, formulations, and uses for modulating gene expression or gene activity in a cell or subject. More specifically, this disclosure relates to activity-generating delivery molecules.

In some aspects, an activity-generating delivery molecule may be composed into a nanoparticle form, or a layered structure or vesicle, or other form of delivery-enhancing composition.

In certain aspects, an activity-generating delivery molecule of this invention may be distinguished by having reduced or insignificant thermotropic or melting transitions.

The molecules and compositions of this disclosure may further be used for delivery of therapeutic, prophylactic, and diagnostic agents such as nucleic acid agents, polynucleotides, peptides, proteins, as well as small molecule compounds and drugs.

The molecules and methods of this invention are useful for delivery of therapeutic agents in forms such as encapsulated within nanoparticles or lamellar vehicles. These forms may include nanoparticles of various diameters, or bilayered or multilayered structures.

### Activity-Generating Delivery Molecules

This invention provides a range of synthetic activity-generating delivery molecules.

A synthetic activity-generating delivery compound of this invention may be prepared by substituting a delivery-enhancing group at both the N-terminus and the C-terminus of an amino acid.

A delivery-enhancing group of this disclosure may include a long chain group, or a lipophilic tail, or a long chain alkenyl, or a substituted variation of any one of the foregoing, where the delivery-enhancing group is unsaturated, and may contain one or more carbon-carbon double bonds.

In some embodiments, a synthetic activity-generating delivery molecule of this invention has a long chain alkenyl group at both the N-terminus and the C-terminus of an amino acid.

In further embodiments, a synthetic activity-generating delivery molecule of this invention has a long chain alkenyl group at both the N-terminus and the C-terminus of an amino acid, so that each terminus of the amino acid is attached to a long chain substituent that has one or more carbon-carbon double bonds.

In additional embodiments, a synthetic activity-generating delivery molecule of this invention has a long chain alkenyl group at both the N-terminus and the C-terminus of an amino acid, so that each terminus of the amino acid is attached to a long chain substituent that has two or more carbon-carbon double bonds.

A delivery-enhancing or long chain group of this disclosure can include an organic group consisting of carbon, oxygen, nitrogen, sulfur, and hydrogen atoms, and having from 12 to 24 carbon atoms, or from 12 to 40 carbon atoms.

In some embodiments, this invention provides a range of activity-generating delivery molecules as shown in Formula I:

R³—(C═O)-Xaa-NH—R⁴ Formula I
wherein
   Xaa is any D- or L-amino acid residue having the general formula

—NR^{N}—CR¹R²—(C═O)—, wherein
R¹ is a non-hydrogen, substituted or unsubstituted side chain of an amino acid;
R², R^{N} are independently hydrogen, or an organic group consisting of carbon, oxygen, nitrogen, sulfur, and hydrogen atoms, and having from 1 to 20 carbon atoms, or C(1-5)alkyl, cycloalkyl, cycloalkylalkyl, C(3-5)alkenyl, C(3-5)alkynyl, C(1-5)alkanoyl, C(1-5)alkanoyloxy, C(1-5)alkoxy, C(1-5)alkoxy-C(1-5)alkyl, C(1-5)alkoxy-C(1-5)alkoxy, C(1-5)alkyl-amino-C(1-5)alkyl-, C(1-5)dialkyl-amino-C(1-5)alkyl-, nitro-C(1-5)alkyl, cyano-C(1-5)alkyl, aryl-C(1-5)alkyl, 4-biphenyl-C(1-5)alkyl, carboxyl, or hydroxyl;
R³—(C═O)— is independently a long chain group which may be derived from a naturally-occurring phospholipid, glycolipid, triacylglycerol, glycerophospholipid, sphingolipid, ceramide, sphingomyelin, cerebroside, or ganglioside, wherein the long chain group contains one or more carbon-carbon double bonds; or a substituted or unsubstituted C(12-24)alkenoyl;
—NH—R⁴ is independently a long chain group which may be derived from a naturally-occurring phospholipid, glycolipid, triacylglycerol, glycerophospholipid, sphingolipid, ceramide, sphingomyelin, cerebroside, or ganglioside, wherein the long chain group contains one or more carbon-carbon double bonds; or a substituted or unsubstituted C(12-24)alkenylamino;
   and salts thereof.

In some embodiments, R¹ is a non-hydrogen, substituted or unsubstituted side chain of an amino acid, where a substituent of a side chain may be an organic group consisting of 1 to 40 atoms selected from hydrogen, carbon, oxygen, nitrogen, and sulfur atoms.

In further embodiments, this invention provides a range of activity-generating delivery molecules as shown in Formula I above wherein:
Xaa is any D- or L-amino acid residue having the general formula

—NR^{N}—CR¹R²—(C═O)—, wherein
R¹ is a non-hydrogen, substituted or unsubstituted side chain of an amino acid;
R², R^{N} are independently hydrogen, or an organic group consisting of carbon, oxygen, nitrogen, sulfur, and hydrogen atoms, and having from 1 to 20 carbon atoms, or C(1-5)alkyl, cycloalkyl, cycloalkylalkyl, C(3-5)alkenyl, C(3-5)alkynyl, C(1-5)alkanoyl, C(1-5)alkanoyloxy, C(1-5)alkoxy, C(1-5)alkoxy-C(1-5)alkyl, C(1-5)alkoxy-C(1-5)alkoxy, C(1-5)alkyl-amino-C(1-5)alkyl-, C(1-5)dialkyl-amino-C(1-5)alkyl-, nitro-C(1-5)alkyl, cyano-C(1-5)alkyl, aryl-C(1-5)alkyl, 4-biphenyl-C(1-5)alkyl, carboxyl, or hydroxyl; R³—(C═O)— is independently a substituted or unsubstituted C(14-24)alkenoyl;
—NH—R⁴ is independently a substituted or unsubstituted C(14-24)alkenylamino; and salts thereof.

In further embodiments, this invention provides a range of activity-generating delivery molecules having the formula R³—(C═O)-Xaa-NH—R⁴, wherein Xaa is any D- or L-amino acid residue, R³—(C═O)— is independently a substituted or unsubstituted C(14-24)alkenoyl; —NH—R⁴ is independently a substituted or unsubstituted C(14-24)alkenylamino; and salts thereof.

An activity-generating delivery molecule of this invention can be neutral, anionic, cationic, zwitterionic, or non-ionic.

As used herein, the physical charge, state or ionicity of a molecule refers to an environment having pH 7, unless otherwise specified.

In some embodiments, this invention provides a range of activity-generating delivery molecules corresponding to Formula I which are represented by the structure

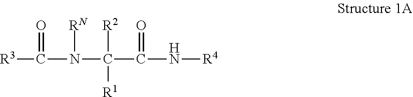

where R¹, R², R^{N}, R³, and R⁴ are defined as above.

In some embodiments, R³ and R⁴ are independently selected groups which impart sufficient lipophilic character or lipophilicity, such as defined by water/octanol partitioning, to provide delivery across a membrane or uptake by a cell.

In certain embodiments, R³ and R⁴ are independently selected long chain groups which impart lipophilic character to provide delivery across a membrane or uptake by a cell.

In some embodiments, R³,R⁴ may independently be C12alkenyl, C13alkenyl, C14alkenyl, C15alkenyl, C16alkenyl, C17alkenyl, C18alkenyl, C19alkenyl, C20alkenyl, C21alkenyl, C22alkenyl, C23alkenyl, or C24alkenyl. In certain embodiments, R³,R⁴ may independently be C(14-24)alkenyl, C(16-24)alkenyl, or C(18-24)alkenyl.

In some embodiments, R³—(C═O)— may independently be C12alkenoyl, C13 alkenoyl, C14alkenoyl, C15alkenoyl, C16alkenoyl, C17alkenoyl, C18alkenoyl, C19alkenoyl, C20alkenoyl, C21alkenoyl, C22alkenoyl, C23alkenoyl, C24alkenoyl. In certain embodiments, R³—(C═O)— may independently be C(14-24)alkenoyl, C(16-24)alkenoyl, or C(18-24)alkenoyl.

In some embodiments, —NH—R⁴ may independently be C12alkenylamino, C13 alkenylamino, C14alkenylamino, C15alkenylamino, C16 alkenylamino, C17alkenylamino, C18alkenylamino, C19 alkenylamino, C20 alkenylamino, C21alkenylamino, C22alkenylamino, C23alkenylamino or C24alkenylamino. In certain embodiments, —NH—R⁴ may independently be C(14-24)alkenylamino, C(16-24)alkenylamino, or C(18-24)alkenylamino.

In some embodiments, R³—(C═O)— may independently be C(12:1)alkenoyl, C(12:2)alkenoyl, or C(12:3)alkenoyl.

In some embodiments, —NH—R⁴ may independently be C(12:1)alkenylamino, C(12:2)alkenylamino, or C(12:3)alkenylamino.

In some embodiments, R³—(C═O)— may independently be C(14:1)alkenoyl, C(14:2)alkenoyl, or C(14:3)alkenoyl, including C(14:1(5))alkenoyl or myristoleic, and C(14:1(9))alkenoyl.

In some embodiments, —NH—R⁴ may independently be C(14:1)alkenylamino, C(14:2)alkenylamino, or C(14:3)alkenylamino, including C(14:1(5))alkenylamino, and C(14:1(9))alkenylamino.

In some embodiments, R³—(C═O)— may independently be C(16:1)alkenoyl, C(16:2)alkenoyl, or C(16:3)alkenoyl, including C(16:1(7))alkenoyl or palmitoleic, and C(16:1(9))alkenoyl.

In some embodiments, —NH—R⁴ may independently be C(16:1)alkenylamino, C(16:2)alkenylamino, or C(16:3)alkenylamino, including C(16:1(7))alkenylamino, and C(16:1(9))alkenylamino.

In some embodiments, R³—(C═O)— may independently be C(18:1)alkenoyl, C(18:2)alkenoyl, or C(18:3)alkenoyl, including C(18:1(3))alkenoyl, C(18:1(5))alkenoyl, C(18:1(7))alkenoyl or cis-vaccenic, C(18:1(9))alkenoyl or oleic, C(18:1(11))alkenoyl, and C(18:1(12))alkenoyl or petroselinic.

In some embodiments, —NH—R⁴ may independently be C(18:1)alkenylamino, C(18:2)alkenylamino, or C(18:3)alkenylamino, including C(18:1(3))alkenylamino, C(18:1(5))alkenylamino, C(18:1(7))alkenylamino, C(18:1(9))alkenylamino, C(18:1(11))alkenylamino, and C(18:1(12))alkenylamino.

In some embodiments, R³—(C═O)— may independently be C(18:2(9,12))alkenoyl, which may be cis,cis-9,12-octadecadienoyl, or C(18:2(9,11))alkenoyl.

In some embodiments, —NH—R⁴ may independently be C(18:2(9,12))alkenylamino, or C(18:2(9,11))alkenylamino.

In some embodiments, R³—(C═O)— may independently be C(18:3(9,12,15))alkenoyl or 9,12,15-octadecatrienoyl.

In some embodiments, —NH—R⁴ may independently be C(18:3(9,12,15))alkenylamino.

In some embodiments, R³—(C═O)— may independently be C(18:3(6,9,12))alkenoyl, or 6,9,12-octadecatrienoyl.

In some embodiments, —NH—R⁴ may independently be C(18:3(6,9,12))alkenylamino.

In some embodiments, R³—(C═O)— may independently be C(18:3(9,11,13))alkenoyl or 9,11,13-octadecatrienoyl.

In some embodiments, —NH—R⁴ may independently be C(18:3(9,11,13))alkenylamino.

In some embodiments, R³—(C═O)— may independently be C(18:4(6,9,12,15))alkenoyl, or C(18:4(9,11,13,15))alkenoyl.

In some embodiments, —NH—R⁴ may independently be C(18:4(6,9,12,15))alkenylamino, or C(18:4(9,11,13,15))alkenylamino.

In some embodiments, R³—(C═O)— may independently be C(20:1(9))alkenoyl, C(20:1(11))alkenoyl, C(22:1(9))alkenoyl, C(22:1(13))alkenoyl, or C(24:1(9))alkenoyl.

In some embodiments, —NH—R⁴ may independently be C(20:1(9))alkenylamino, C(20:1(11))alkenylamino, C(22:1(9))alkenylamino, C(22:1(13))alkenylamino, or C(24:1(9))alkenylamino.

In some embodiments, R³—(C═O)— may independently be C(20:2(8,11))alkenoyl or 8,11-icosadienoyl, C(20:2(5,8))alkenoyl, or C(20:2(11,14))alkenoyl.

In some embodiments, —NH—R⁴ may independently be C(20:2(8,11))alkenylamino, C(20:2(5,8))alkenylamino, or C(20:2(11,14))alkenylamino.

In some embodiments, R³—(C═O)— may independently be C(20:3(5,8,11))alkenoyl or 5,8,11-icosatrienoyl.

In some embodiments, —NH—R⁴ may independently be C(20:3(5,8,11))alkenylamino.

In some embodiments, R³—(C═O)— may independently be C(20:4(5,8,11,14))alkenoyl, or C(20:4(7,10,13,16))alkenoyl.

In some embodiments, —NH—R⁴ may independently be C(20:4(5,8,11,14))alkenylamino, or C(20:4(7,10,13,16))alkenylamino.

In some embodiments, R³—(C═O)— may independently be C(20:5(5,8,11,14,17))alkenoyl.

In some embodiments, —NH—R⁴ may independently be C(20:5(5,8,11,14,17))alkenylamino.

In some embodiments, R³—(C═O)— may independently be C(20:6(4,7,10,13,16,19))alkenoyl.

In some embodiments, —NH—R⁴ may independently be C(20:6(4,7,10,13,16,19))alkenylamino.

In some embodiments, R³ and R⁴ may independently be one of the following structures:

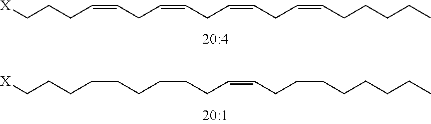

In certain embodiments, R³ and R⁴ may independently be derived from fatty acid-like tails such as tails from oleic acid (C18:1, double bond at carbon 9)alkenyl, linoleic acid (C18:2, double bond at carbon 9 or 12)alkenyl, linonenic acid (C18:3, double bond at carbon 9, 12, or 15)alkenyl, arachidonic acid (C20:4, double bond at carbon 5, 8, 11, or 14)alkenyl, and eicosapentaenoic acid (C20:5, double bond at carbon 5, 8, 11, 14, or 17)alkenyl. Other examples of fatty acid-like tails are found at Donald Voet and Judith Voet, Biochemistry, 3rd Edition (2005), p. 383.

### Amino Acid Definition

As used herein, the term “amino acid” includes naturally-occurring and non-naturally occurring amino acids. Thus, an activity-generating delivery molecule of this invention can be can be made from a genetically encoded amino acid, a naturally occurring non-genetically encoded amino acid, or a synthetic amino acid.

Examples of amino acids include Ala, Arg, Asn, Asp, Cys, Gln, Glu, Gly, His, Ile, Leu, Lys, Met, Phe, Pro, Ser, Thr, Tip, Tyr, and Val.

Examples of amino acids include azetidine, 2-aminooctadecanoic acid, 2-aminoadipic acid, 3-aminoadipic acid, 2,2-diaminoacetic acid, 2,3-diaminopropionic acid, 2-aminobutyric acid, 4-aminobutyric acid, 2,3-diaminobutyric acid, 2,4-diaminobutyric acid, 2-aminoisobutyric acid, 4-aminoisobutyric acid, 2-aminopimelic acid, 2,2′-diaminopimelic acid, 6-aminohexanoic acid, 6-aminocaproic acid, 2-aminoheptanoic acid, desmosine, ornithine, citrulline, N-methylisoleucine, norleucine, tert-leucine, phenylglycine, t-butylglycine, N-methylglycine, sacrosine, N-ethylglycine, cyclohexylglycine, 4-oxo-cyclohexylglycine, N-ethylasparagine, cyclohexylalanine, t-butylalanine, naphthylalanine, pyridylalanine, 3-chloroalanine, 3-benzothienylalanine, 4-halophenylalanine, 4-chlorophenylalanine, 2-fluorophenylalanine, 3-fluorophenylalanine, 4-fluorophenylalanine, penicillamine, 2-thienylalanine, methionine, methionine sulfoxide, homoarginine, norarginine, nor-norarginine, N-acetyllysine, 4-aminophenylalanine, N-methylvaline, homocysteine, homoserine, hydroxylysine, allo-hydroxylysine, 3-hydroxyproline, 4-hydroxyproline, isodesmosine, allo-isoleucine, 6-N-methyllysine, norvaline, O-allyl-serine, O-allyl-threonine, alpha-aminohexanoic acid, alpha-aminovaleric acid, and pyroglutamic acid.

As used herein, the term “amino acid” includes alpha- and beta-amino acids.

Other amino acid residues can be found in Fasman, CRC Practical Handbook of Biochemistry and Molecular Biology, CRC Press, Inc. (1989).

In general, a compound may contain one or more chiral centers. Compounds containing one or more chiral centers may include those described as an “isomer,” a “stereoisomer,” a “diastereomer,” an “enantiomer,” an “optical isomer,” or as a “racemic mixture.” Conventions for stereochemical nomenclature, for example the stereoisomer naming rules of Cahn, Ingold and Prelog, as well as methods for the determination of stereochemistry and the separation of stereoisomers are known in the art. See, for example, Michael B. Smith and Jerry March, March's Advanced Organic Chemistry, 5th edition, 2001. The compounds and structures of this disclosure are meant to encompass all possible isomers, stereoisomers, diastereomers, enantiomers, and/or optical isomers that would be understood to exist for the specified compound or structure, including any mixture, racemic or otherwise, thereof.

In particular, the long chain groups R³—(C═O)— and —NH—R⁴ may be any combination of cis or trans isomers that would be understood to exist, including any mixture thereof.

Names for Activity-Generating Delivery Molecules of this Invention

As used herein, the designation “(18:1(9))-norArg-(18:1(9)),” for example, refers to (C17:1(9)alkenyl)-(C═O)-norArg-NH—(C18:1(9)alkenyl), which is the same as (C18:1(9)alkenoyl)-norArg-NH—(C18:1(9)alkenyl), which is the same as (C18:1(9)alkenoyl)-norArg-(C18:1(9)alkenylamino). In this naming, the number in the inner parenthesis, for example the 9 in 18:1(9), refers to the position of a double bond counting from the (C═O), or counting from the carbon atom attached to the NH as the number one position.

### DAP Activity-Generating Delivery Molecules

Examples of an activity-generating delivery molecule of this invention include R³—(C═O)-Xaa-NH—R⁴ wherein R³ and R⁴ are as defined above, and Xaa is a D- or L-diaminoproprionic acid residue.

Examples of an activity-generating delivery molecule of this invention include R³—(C═O)-DAP—NH—R⁴ where DAP is a D- or L-diaminoproprionic acid residue, and R³ and R⁴ are substituted or unsubstituted C(14-24)alkenyl, and salts thereof.

Examples of an activity-generating delivery molecule include R³—(C═O)-DAP—NH—R⁴ where DAP is a D- or L-diaminoproprionic acid residue, R³—(C═O)— is (18:1)oleoyl, and —NH—R⁴ is (18:1)alkenylamino, where (18:1)alkenylamino includes C(18:1(3))alkenylamino, C(18:1(5))alkenylamino, C(18:1(7))alkenylamino, C(18:1(9))alkenylamino, C(18:1(11))alkenylamino, and C(18:1 (12))alkenylamino.

Examples of an activity-generating delivery molecule include

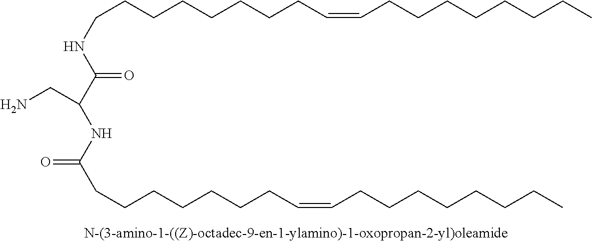 [SMILES: CCCCCCCC/C=C\CCCCCCCCNC(=O)C(CN)NC(=O)CCCCCCC/C=C\CCCCCCCC]

Examples of an activity-generating delivery molecule include R³—(C═O)-DAP—NH—R⁴ where DAP is a D- or L-diaminoproprionic acid residue, R³—(C═O)— is (18:1)oleoyl, and —NH—R⁴ is (18:2)alkenylamino, where (18:2)alkenylamino includes C(18:2(9,12))alkenylamino.

Examples of an activity-generating delivery molecule include

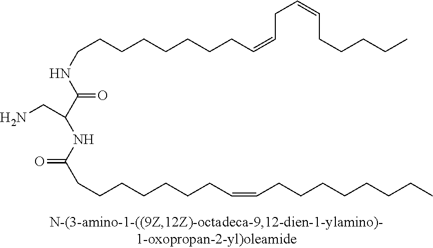 [SMILES: CCCCC/C=C\C/C=C\CCCCCCCCNC(=O)C(CN)NC(=O)CCCCCCC/C=C\CCCCCCCC]

Examples of an activity-generating delivery molecule include R³—(C═O)-DAP—NH—R⁴ where DAP is a D- or L-diaminoproprionic acid residue, R³—(C═O)— is (18:2)oleoyl, and —NH—R⁴ is (18:1)alkenylamino as defined above.

Examples of an activity-generating delivery molecule include

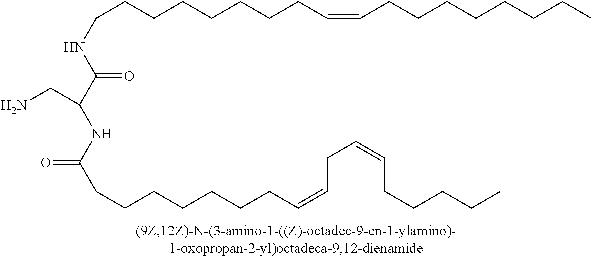 [SMILES: CCCCC/C=C\C/C=C\CCCCCCCC(=O)NC(CN)C(=O)NCCCCCCCC/C=C\CCCCCCCC]

Examples of an activity-generating delivery molecule include R³—(C═O)-DAP—NH—R⁴ where DAP is a D- or L-diaminoproprionic acid residue, R³—(C═O)— is (18:2)oleoyl, and —NH—R⁴ is (18:2)alkenylamino.

Examples of an activity-generating delivery molecule include

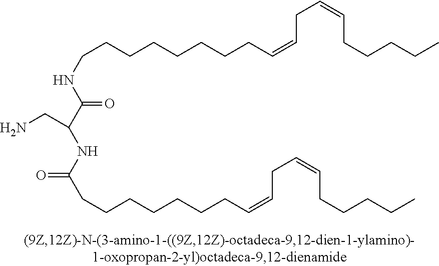 [SMILES: CCCCC/C=C\C/C=C\CCCCCCCCNC(=O)C(CN)NC(=O)CCCCCCC/C=C\C/C=C\CCCCC]

Examples of an activity-generating delivery molecule include (18:1(3))-DAP-(18:1(3)), (18:1(5))-DAP-(18:1(5)), (18:1(7))-DAP-(18:1(7)), (18:1(9))-DAP-(18:1(9)), (18:1(11))-DAP-(18:1(11)), (18:1(12))-DAP-(18:1(12)), (18:1(3))-DAP-(18:1(5)), (18:1(3))-DAP-(18:1(7)), (18:1(3))-DAP-(18:1(9)), (18:1(3))-DAP-(18:1(11)), (18:1(3))-DAP-(18:1(12)), (18:1(5))-DAP-(18:1(7)), (18:1(5))-DAP-(18:1(9)), (18:1(5))-DAP-(18:1(11)), (18:1(5))-DAP-(18:1(12)), (18:1(7))-DAP-(18:1(9)), (18:1(7))-DAP-(18:1(11)), (18:1(7))-DAP-(18:1(12)), (18:1(9))-DAP-(18:1(11)), (18:1(9))-DAP-(18:1(12)), and (18:1(11))-DAP-(18:1(12)).

Examples of an activity-generating delivery molecule include (18:1(3))-DAP-(18:2(9,12)), (18:1(5))-DAP-(18:2(9,12)), (18:1(7))-DAP-(18:2(9,12)), (18:1(9))-DAP-(18:2(9,12)), (18:1(11))-DAP-(18:2(9,12)), and (18:1(12))-DAP-(18:2(9,12)).

Examples of an activity-generating delivery molecule include (18:2(9,12))-DAP-(18:1(3)), (18:2(9,12))-DAP-(18:1(5)), (18:2(9,12))-DAP-(18:1(7)), (18:2(9,12))-DAP-(18:1(9)), (18:2(9,12))-DAP-(18:1(11)), and (18:2(9,12))-DAP-(18:1(12)).

Examples of an activity-generating delivery molecule include (18:2(9,12))-DAP-(18:2(9,12)).

Any of the foregoing activity-generating delivery molecules wherein Xaa is a D- or L-diaminoproprionic acid residue can have the side chain amino group of the residue quaternized by hydrogen to form —NH₃⁺ or by one or more methyl, ethyl, propyl or butyl groups (“R” groups) to form —NH₂R⁺, —NHR₂⁺, or —NR₃⁺ all of which are side chain quaternary ammonium groups or cationic forms.

### DAB Activity-Generating Delivery Molecules

Examples of an activity-generating delivery molecule of this invention include R³—(C═O)-Xaa-NH—R⁴ wherein R³ and R⁴ are as defined above, and Xaa is a D- or L-2,4-diaminobutyric acid residue.

Examples of an activity-generating delivery molecule of this invention include R³—(C═O)-DAB—NH—R⁴ where DAB is a D- or L-2,4-diaminobutyric acid residue, and R³ and R⁴ are substituted or unsubstituted C(14-24)alkenyl, and salts thereof.

Examples of an activity-generating delivery molecule include R³—(C═O)-DAB—NH—R⁴ where DAB is a D- or L-2,4-diaminobutyric acid residue, R³—(C═O)— is (18:1)oleoyl, and —NH—R⁴ is (18:1)alkenylamino as defined above.

Examples of an activity-generating delivery molecule include

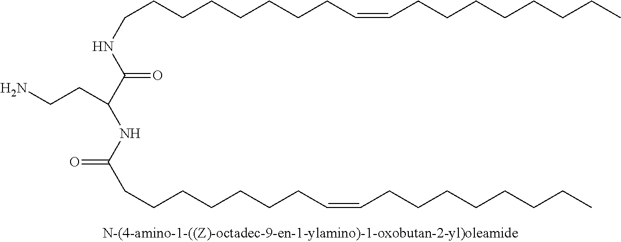 [SMILES: CCCCCCCC/C=C\CCCCCCCCNC(=O)C(CCN)NC(=O)CCCCCCC/C=C\CCCCCCCC]

Examples of an activity-generating delivery molecule include R³—(C═O)-DAB—NH—R⁴ where DAB is a D- or L-2,4-diaminobutyric acid residue, R³—(C═O)— is (18:1)oleoyl, and —NH—R⁴ is (18:2)alkenylamino, as defined above.

Examples of an activity-generating delivery molecule include

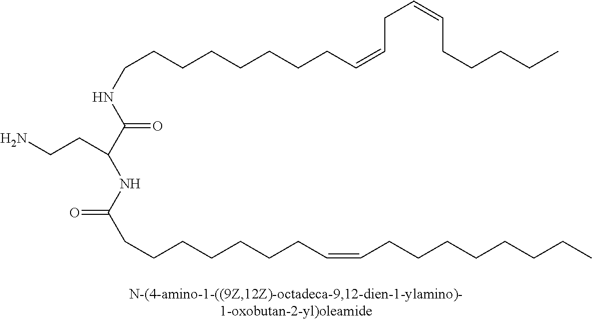 [SMILES: CCCCC/C=C\C/C=C\CCCCCCCCNC(=O)C(CCN)NC(=O)CCCCCCC/C=C\CCCCCCCC]

Examples of an activity-generating delivery molecule include R³—(C═O)-DAB—NH—R⁴ where DAB is a D- or L-2,4-diaminobutyric acid residue, R³—(C═O)— is (18:2)oleoyl, and —NH—R⁴ is (18:1)alkenylamino.

Examples of an activity-generating delivery molecule include

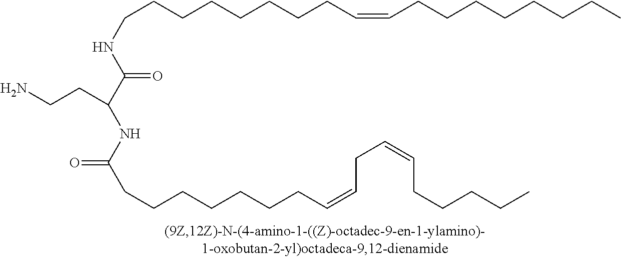 [SMILES: CCCCC/C=C\C/C=C\CCCCCCCC(=O)NC(CCN)C(=O)NCCCCCCCC/C=C\CCCCCCCC]

Examples of an activity-generating delivery molecule include R³—(C═O)-DAB—NH—R⁴ where DAB is a D- or L-2,4-diaminobutyric acid residue, R³—(C═O)— is (18:2)oleoyl, and —NH—R⁴ is (18:2)alkenylamino, which is also referred to herein as C(18:2)oleoyl-DAB-C(18:2)alkenylamino, or C18:2-DAB-C18:2.

Examples of an activity-generating delivery molecule include

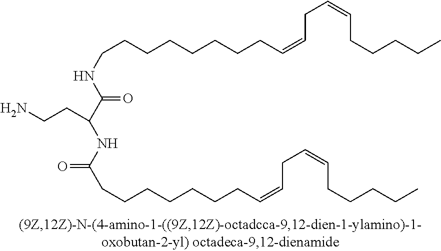 [SMILES: CCCCC/C=C\C/C=C\CCCCCCCCNC(=O)C(CCN)NC(=O)CCCCCCC/C=C\C/C=C\CCCCC]

An ionic form of this molecule is C(18:2)oleoyl-DAB(NH₃⁺Cl⁻)—C(18:2)alkenylamino, or C18:2-DAB(NH₃⁺Cl⁻)—C18:2.

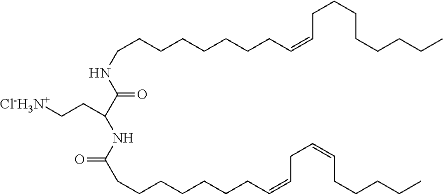

Examples of an activity-generating delivery molecule include (18:1(3))-DAB-(18:1(3)), (18:1(5))-DAB-(18:1(5)), (18:1(7))-DAB-(18:1(7)), (18:1(9))-DAB-(18:1(9)), (18:1(11))-DAB-(18:1(11)), (18:1(12))-DAB-(18:1(12)), (18:1(3))-DAB-(18:1(5)), (18:1(3))-DAB-(18:1(7)), (18:1(3))-DAB-(18:1(9)), (18:1(3))-DAB-(18:1(11)), (18:1(3))-DAB-(18:1(12)), (18:1(5))-DAB-(18:1(7)), (18:1(5))-DAB-(18:1(9)), (18:1(5))-DAB-(18:1(11)), (18:1(5))-DAB-(18:1(12)), (18:1(7))-DAB-(18:1(9)), (18:1(7))-DAB-(18:1(11)), (18:1(7))-DAB-(18:1(12)), (18:1(9))-DAB-(18:1(11)), (18:1(9))-DAB-(18:1(12)), and (18:1(11))-DAB-(18:1(12)).

Examples of an activity-generating delivery molecule include (18:1(3))-DAB-(18:2(9,12)), (18:1(5))-DAB-(18:2(9,12)), (18:1(7))-DAB-(18:2(9,12)), (18:1(9))-DAB-(18:2(9,12)), (18:1(11))-DAB-(18:2(9,12)), and (18:1(12))-DAB-(18:2(9,12)).

Examples of an activity-generating delivery molecule include (18:2(9,12))-DAB-(18:1(3)), (18:2(9,12))-DAB-(18:1(5)), (18:2(9,12))-DAB-(18:1(7)), (18:2(9,12))-DAB-(18:1(9)), (18:2(9,12))-DAB-(18:1(11)), and (18:2(9,12))-DAB-(18:1(12)).

Examples of an activity-generating delivery molecule include (18:2(9,12))-DAB-(18:2(9,12)).

Any of the foregoing activity-generating delivery molecules wherein Xaa is a D- or L-2,4-diaminobutyric acid residue can have the side chain amino group of the residue quaternized by hydrogen to form —NH₃⁺ or by one or more methyl, ethyl, propyl or butyl groups (“R” groups) to form —NH₂R^{|}, —NHR₂^{|}, or —NR₃^{|}, all of which are side chain quaternary ammonium groups or cationic forms.

### DAA Activity-Generating Delivery Molecules

Examples of an activity-generating delivery molecule of this invention include R³—(C═O)-Xaa-NH—R⁴ wherein R³ and R⁴ are as defined above, and Xaa is a D- or L-2,2-diaminoacetic acid residue.

Examples of an activity-generating delivery molecule of this invention include R³—(C═O)-DAA-NH—R⁴ where DAA is a D- or L-2,2-diaminoacetic acid residue, and R³ and R⁴ are substituted or unsubstituted C(14-24)alkenyl, and salts thereof.

Examples of an activity-generating delivery molecule include R³—(C═O)-DAA-NH—R⁴ where DAA is a D- or L-2,2-diaminoacetic acid residue, R³—(C═O)— is (18:1)olcoyl, and —NH—R⁴ is (18:1)alkenylamino, where (18:1)alkenylamino includes C(18:1(3))alkenylamino, C(18:1(5))alkenylamino, C(18:1(7))alkenylamino, C(18:1(9))alkenylamino, C(18:1(11))alkenylamino, and C(18:1(12))alkenylamino.

Examples of an activity-generating delivery molecule include

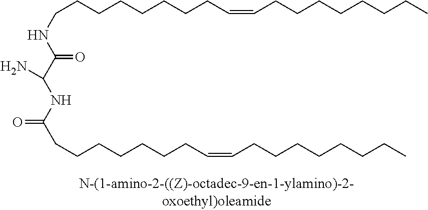 [SMILES: CCCCCCCC/C=C\CCCCCCCCNC(=O)C(N)NC(=O)CCCCCCC/C=C\CCCCCCCC]

Examples of an activity-generating delivery molecule include R³—(C═O)-DAA-NH—R⁴ where DAA is a D- or L-2,2-diaminoacetic acid residue, R³—(C═O)— is (18:1)oleoyl, and —NH—R⁴ is (18:2)alkenylamino, where (18:2)alkenylamino includes C(18:2(9,12)) alkenylamino or cis,cis-9,12-octadecadienylamino.

Examples of an activity-generating delivery molecule include

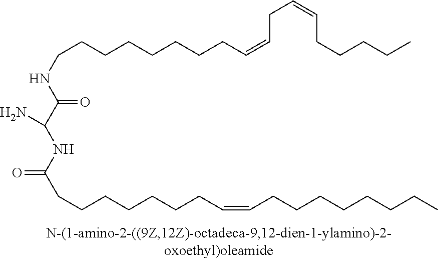 [SMILES: CCCCC/C=C\C/C=C\CCCCCCCCNC(=O)C(N)NC(=O)CCCCCCC/C=C\CCCCCCCC]

Examples of an activity-generating delivery molecule include R³—(C═O)-DAA-NH—R⁴ where DAA is a D- or L-2,2-diaminoacetic acid residue, R³—(C═O)— is (18:2)oleoyl, and —NH—R⁴ is (18:1)alkenylamino.

Examples of an activity-generating delivery molecule include

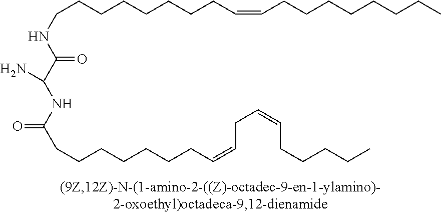 [SMILES: CCCCC/C=C\C/C=C\CCCCCCCC(=O)NC(N)C(=O)NCCCCCCCC/C=C\CCCCCCCC]

Examples of an activity-generating delivery molecule include R³—(C═O)-DAA-NH—R⁴ where DAA is a D- or L-2,2-diaminoacetic acid residue, R³—(C═O)— is (18:2)oleoyl, and —NH—R⁴ is (18:2)alkenylamino.

Examples of an activity-generating delivery molecule include

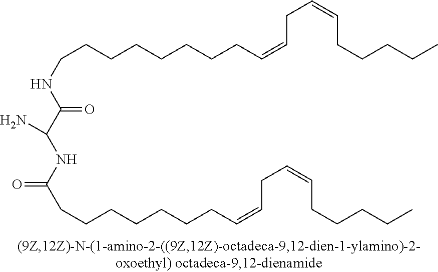 [SMILES: CCCCC/C=C\C/C=C\CCCCCCCCNC(=O)C(N)NC(=O)CCCCCCC/C=C\C/C=C\CCCCC]

Examples of an activity-generating delivery molecule include (18:1(3))-DAA-(18:1(3)), (18:1(5))-DAA-(18:1(5)), (18:1(7))-DAA-(18:1(7)), (18:1(9))-DAA-(18:1(9)), (18:1(11))-DAA-(18:1(11)), (18:1(12))-DAA-(18:1(12)), (18:1(3))-DAA-(18:1(5)), (18:1(3))-DAA-(18:1(7)), (18:1(3))-DAA-(18:1(9)), (18:1(3))-DAA-(18:1(11)), (18:1(3))-DAA-(18:1(12)), (18:1(5))-DAA-(18:1(7)), (18:1(5))-DAA-(18:1(9)), (18:1(5))-DAA-(18:1(11)), (18:1(5))-DAA-(18:1(12)), (18:1(7))-DAA-(18:1(9)), (18:1(7))-DAA-(18:1(11)), (18:1(7))-DAA-(18:1(12)), (18:1(9))-DAA-(18:1(11)), (18:1(9))-DAA-(18:1(12)), and (18:1(11))-DAA-(18:1(12)).

Examples of an activity-generating delivery molecule include (18:1(3))-DAA-(18:2(9,12)), (18:1(5))-DAA-(18:2(9,12)), (18:1(7))-DAA-(18:2(9,12)), (18:1(9))-DAA-(18:2(9,12)), (18:1(11))-DAA-(18:2(9,12)), and (18:1(12))-DAA-(18:2(9,12)).

Examples of an activity-generating delivery molecule include (18:2(9,12))-DAA-(18:1(3)), (18:2(9,12))-DAA-(18:1(5)), (18:2(9,12))-DAA-(18:1(7)), (18:2(9,12))-DAA-(18:1(9)), (18:2(9,12))-DAA-(18:1(11)), and (18:2(9,12))-DAA-(18:1(12)).

Examples of an activity-generating delivery molecule include (18:2(9,12))-DAA-(18:2(9,12)).

Any of the foregoing activity-generating delivery molecules wherein Xaa is a D- or L-2,2-diaminoacetic acid residue can have the side chain amino group of the residue quaternized by hydrogen to form a —NH₃⁺, or by one or more methyl, ethyl, propyl or butyl groups (“R” groups) to form —NH₂R⁺, —NHR₂⁺, or —NR₃⁺, all of which are side chain quaternary ammonium groups or cationic forms.

### Orn Activity-Generating Delivery Molecules

Examples of an activity-generating delivery molecule of this invention include R³—(C═O)-Xaa-NH—R⁴ wherein R³ and R⁴ are as defined above, and Xaa is D- or L-ornithine.

Examples of an activity-generating delivery molecule of this invention include R³—(C═O)—Orn-NH—R⁴ where Orn is D- or L-ornithine, and R³ and R⁴ are substituted or unsubstituted C(14-24)alkenyl, and salts thereof.

Examples of an activity-generating delivery molecule include R³—(C═O)-Orn-NH—R⁴ where Orn is D- or L-ornithine, R³—(C═O)— is (18:1)oleoyl, and —NH—R⁴ is (18:1)alkenylamino as defined above.

Examples of an activity-generating delivery molecule include

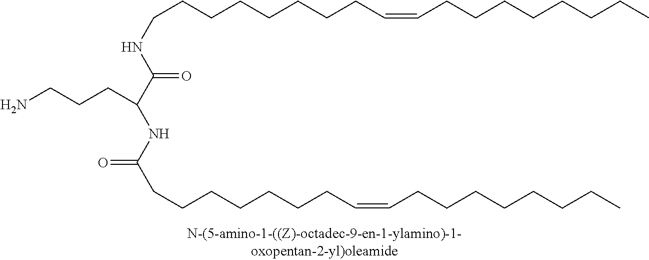 [SMILES: CCCCCCCC/C=C\CCCCCCCCNC(=O)C(CCCN)NC(=O)CCCCCCC/C=C\CCCCCCCC]

Examples of an activity-generating delivery molecule include R³—(C═O)-Orn-NH—R⁴ where Orn is D- or L-ornithine, R³—(C═O)— is (18:1)oleoyl, and —NH—R⁴ is (18:2)alkenylamino as defined above.

Examples of an activity-generating delivery molecule include

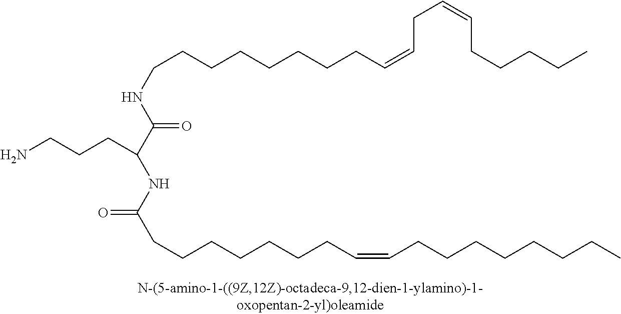 [SMILES: CCCCC/C=C\C/C=C\CCCCCCCCNC(=O)C(CCCN)NC(=O)CCCCCCC/C=C\CCCCCCCC]

Examples of an activity-generating delivery molecule include R³—(C═O)-Orn-NH—R⁴ where Orn is D- or L-ornithine, R³—(C═O)— is (18:2)oleoyl, and —NH—R⁴ is (18:1)alkenylamino.

Examples of an activity-generating delivery molecule include

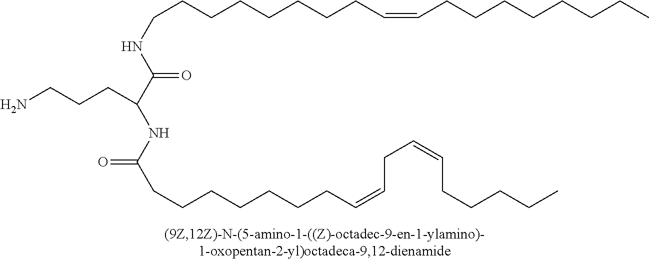 [SMILES: CCCCC/C=C\C/C=C\CCCCCCCC(=O)NC(CCCN)C(=O)NCCCCCCCC/C=C\CCCCCCCC]

Examples of an activity-generating delivery molecule include R³—(C═O)-Orn-NH—R⁴ where Orn is D- or L-ornithine, R³—(C═O)— is (18:2)oleoyl, and —NH—R⁴ is (18:2)alkenylamino.

Examples of an activity-generating delivery molecule include

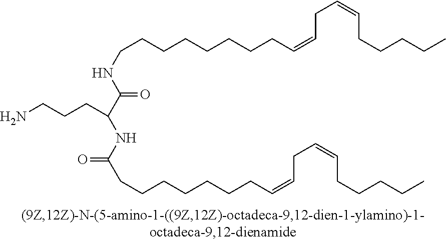 [SMILES: CCCCC/C=C\C/C=C\CCCCCCCCNC(=O)C(CCCN)NC(=O)CCCCCCC/C=C\C/C=C\CCCCC]

Examples of an activity-generating delivery molecule include (18:1(3))-Orn-(18:1(3)), (18:1(5))-Orn-(18:1(5)), (18:1(7))-Orn-(18:1(7)), (18:1(9))-Orn-(18:1(9)), (18:1(11))-Orn-(18:1(11)), (18:1(12))-Orn-(18:1(12)), (18:1(3))-Orn-(18:1(5)), (18:1(3))-Orn-(18:1(7)), (18:1(3))-Orn-(18:1(9)), (18:1(3))-Orn-(18:1(11)), (18:1(3))-Orn-(18:1(12)), (18:1(5))-Orn-(18:1(7)), (18:1(5))-Orn-(18:1(9)), (18:1(5))-Orn-(18:1(11)), (18:1(5))-Orn-(18:1(12)), (18:1(7))-Orn-(18:1(9)), (18:1(7))-Orn-(18:1(11)), (18:1(7))-Orn-(18:1(12)), (18:1(9))-Orn-(18:1(11)), (18:1(9))-Orn-(18:1(12)), and (18:1(11))-Orn-(18:1(12)).

Examples of an activity-generating delivery molecule include (18:1(3))-Orn-(18:2(9,12)), (18:1(5))-Orn-(18:2(9,12)), (18:1(7))-Orn-(18:2(9,12)), (18:1(9))-Orn-(18:2(9,12)), (18:1(11))-Orn-(18:2(9,12)), and (18:1(12))-Orn-(18:2(9,12)).

Examples of an activity-generating delivery molecule include (18:2(9,12))-Orn-(18:1(3)), (18:2(9,12))-Orn-(18:1(5)), (18:2(9,12))-Orn-(18:1(7)), (18:2(9,12))-Orn-(18:1(9)), (18:2(9,12))-Orn-(18:1(11)), and (18:2(9,12))-Orn-(18:1(12)).

Examples of an activity-generating delivery molecule include (18:2(9,12))-Orn-(18:2(9,12)).

Any of the foregoing activity-generating delivery molecules wherein Xaa is D- or L-ornithine can have the side chain amino group of the ornithine quaternized by hydrogen to form —NH₃⁺, or by one or more methyl, ethyl, propyl or butyl groups (“R” groups) to form —NH₂R^{|}, —NHR₂^{|}, or —NR₃^{|}, all of which are side chain quaternary ammonium groups or cationic forms.

### Lys Activity-Generating Delivery Molecules

Examples of an activity-generating delivery molecule of this invention include R³—(C═O)-Xaa-NH—R⁴ wherein R³ and R⁴ are as defined above, and Xaa is D- or L-lysine.

Examples of an activity-generating delivery molecule of this invention include R³—(C═O)-Lys-NH—R⁴ where Lys is D- or L-lysine, and R³ and R⁴ are substituted or unsubstituted C(14-24)alkenyl, and salts thereof.

Examples of an activity-generating delivery molecule include R³—(C═O)-Lys-NH—R⁴ where Lys is D- or L-lysine, R³—(C═O)— is (18:1)oleoyl, and —NH—R⁴ is (18:1)alkenylamino as defined above.

Examples of an activity-generating delivery molecule include

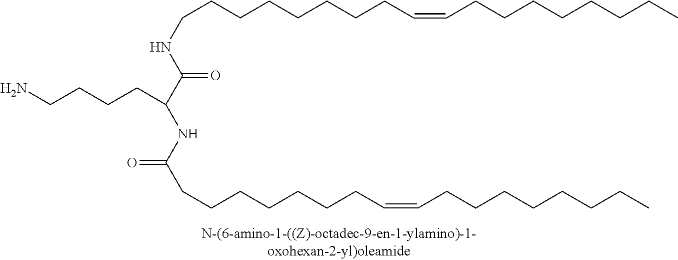 [SMILES: CCCCCCCC/C=C\CCCCCCCCNC(=O)C(CCCCN)NC(=O)CCCCCCC/C=C\CCCCCCCC]

Examples of an activity-generating delivery molecule include R³—(C═O)-Lys-NH—R⁴ where Lys is D- or L-lysine, R³—(C═O)— is (18:1)oleoyl, and —NH—R⁴ is (18:2)alkenylamino as defined above.

Examples of an activity-generating delivery molecule include

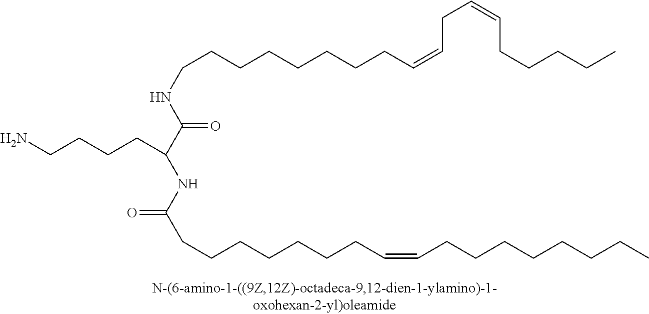 [SMILES: CCCCC/C=C\C/C=C\CCCCCCCCNC(=O)C(CCCCN)NC(=O)CCCCCCC/C=C\CCCCCCCC]

Examples of an activity-generating delivery molecule include R³—(C═O)-Lys-NH—R⁴ where Lys is D- or L-lysine, R³—(C═O)— is (18:2)oleoyl, and —NH—R⁴ is (18:1)alkenylamino.

Examples of an activity-generating delivery molecule include

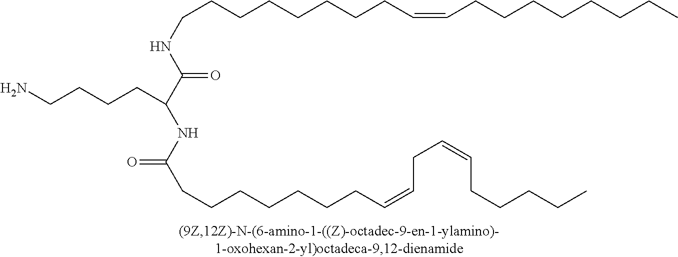 [SMILES: CCCCC/C=C\C/C=C\CCCCCCCC(=O)NC(CCCCN)C(=O)NCCCCCCCC/C=C\CCCCCCCC]

Examples of an activity-generating delivery molecule include R³—(C═O)-Lys-NH—R⁴ where Lys is D- or L-lysine, R³—(C═O)— is (18:2)oleoyl, and —NH—R⁴ is (18:2)alkenylamino.

Examples of an activity-generating delivery molecule include

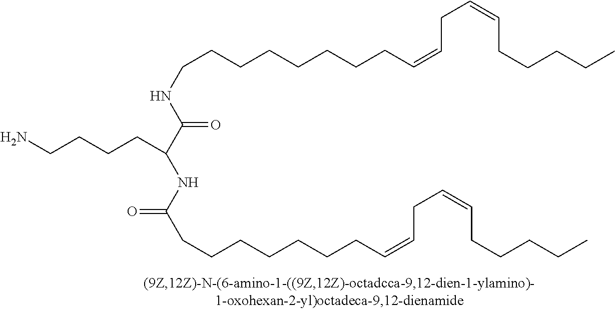 [SMILES: CCCCC/C=C\C/C=C\CCCCCCCCNC(=O)C(CCCCN)NC(=O)CCCCCCC/C=C\C/C=C\CCCCC]

Examples of an activity-generating delivery molecule include (18:1(3))-Lys-(18:1(3)), (18:1(5))-Lys-(18:1(5)), (18:1(7))-Lys-(18:1(7)), (18:1(9))-Lys-(18:1(9)), (18:1(11))-Lys-(18:1(11)), (18:1(12))-Lys-(18:1(12)), (18:1(3))-Lys-(18:1(5)), (18:1(3))-Lys-(18:1(7)), (18:1(3))-Lys-(18:1(9)), (18:1(3))-Lys-(18:1(11)), (18:1(3))-Lys-(18:1(12)), (18:1(5))-Lys-(18:1(7)), (18:1(5))-Lys-(18:1(9)), (18:1(5))-Lys-(18:1(11)), (18:1(5))-Lys-(18:1(12)), (18:1(7))-Lys-(18:1(9)), (18:1(7))-Lys-(18:1(11)), (18:1(7))-Lys-(18:1(12)), (18:1(9))-Lys-(18:1(11)), (18:1(9))-Lys-(18:1(12)), and (18:1(11))-Lys-(18:1(12)).

Examples of an activity-generating delivery molecule include (18:1(3))-Lys-(18:2(9,12)), (18:1(5))-Lys-(18:2(9,12)), (18:1(7))-Lys-(18:2(9,12)), (18:1(9))-Lys-(18:2(9,12)), (18:1(11))-Lys-(18:2(9,12)), and (18:1(12))-Lys-(18:2(9,12)).

Examples of an activity-generating delivery molecule include (18:2(9,12))-Lys-(18:1(3)), (18:2(9,12))-Lys-(18:1(5)), (18:2(9,12))-Lys-(18:1(7)), (18:2(9,12))-Lys-(18:1(9)), (18:2(9,12))-Lys-(18:1(11)), and (18:2(9,12))-Lys-(18:1(12)).

Examples of an activity-generating delivery molecule include (18:2(9,12))-Lys-(18:2(9,12)).

Any of the foregoing activity-generating delivery molecules wherein Xaa is D- or L-lysine can have the side chain amino group of the lysine quaternized by hydrogen to form or by one or more methyl, ethyl, propyl or butyl groups (“R” groups) to form —NHR₂⁺, or —NR₃⁺, all of which are side chain quaternary ammonium groups or cationic forms.

### NorArg Activity-Generating Delivery Molecules

Examples of an activity-generating delivery molecule of this invention include R³—(C═O)-Xaa-NH—R⁴ wherein R³ and R⁴ are as defined above, and Xaa is D- or L-norarginine.

Examples of an activity-generating delivery molecule of this invention include R³—(C═O)-norArg-NH—R⁴ where norArg is D- or L-norarginine, and R³ and R⁴ are substituted or unsubstituted C(14-24)alkenyl, and salts thereof.

Examples of an activity-generating delivery molecule include R³—(C═O)-norArg-NH—R⁴ where norArg is D- or L-norarginine, R³—(C═O)— is (18:1)oleoyl, and —NH—R⁴ is (18:1)alkenylamino as defined above.

Examples of an activity-generating delivery molecule include

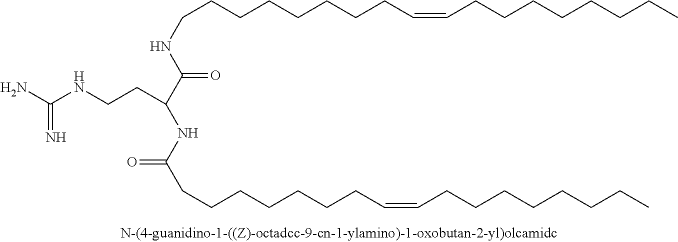 [SMILES: CCCCCCCC/C=C\CCCCCCCCNC(=O)C(CCNC(=N)N)NC(=O)CCCCCCC/C=C\CCCCCCCC]

Examples of an activity-generating delivery molecule include R³—(C═O)-norArg-NH—R⁴ where norArg is D- or L-norarginine, R³—(C═O)— is (18:1)oleoyl, and —NH—R⁴ is (18:2)alkenylamino as defined above.

Examples of an activity-generating delivery molecule include

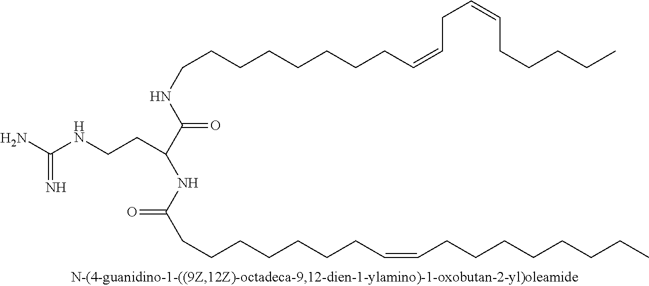 [SMILES: CCCCC/C=C\C/C=C\CCCCCCCCNC(=O)C(CCNC(=N)N)NC(=O)CCCCCCC/C=C\CCCCCCCC]

Examples of an activity-generating delivery molecule include R³—(C═O)-norArg-NH—R⁴ where norArg is D- or L-norarginine, R³—(C═O)— is (18:2)oleoyl, and —NH—R⁴ is (18:1)alkenylamino.

Examples of an activity-generating delivery molecule include

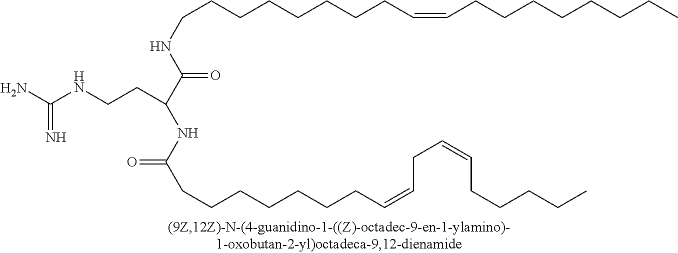 [SMILES: CCCCC/C=C\C/C=C\CCCCCCCC(=O)NC(CCNC(=N)N)C(=O)NCCCCCCCC/C=C\CCCCCCCC]

Examples of an activity-generating delivery molecule include R³—(C═O)-norArg-NH—R⁴ where norArg is D- or L-norarginine, R³—(C═O)— is (18:2)oleoyl, and —NH—R⁴ is (18:2)alkenylamino.

Examples of an activity-generating delivery molecule include

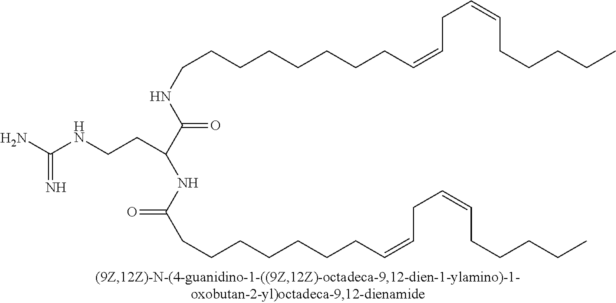 [SMILES: CCCCC/C=C\C/C=C\CCCCCCCCNC(=O)C(CCNC(=N)N)NC(=O)CCCCCCC/C=C\C/C=C\CCCCC]

Examples of an activity-generating delivery molecule include (18:1(3))-norArg-(18:1(3)), (18:1(5))-norArg-(18:1(5)), (18:1(7))-norArg-(18:1(7)), (18:1(9))-norArg-(18:1(9)), (18:1(11))-norArg-(18:1(11)), (18:1(12))-norArg-(18:1(12)), (18:1(3))-norArg-(18:1(5)), (18:1(3))-norArg-(18:1(7)), (18:1(3))-norArg-(18:1(9)), (18:1(3))-norArg-(18:1(11)), (18:1(3))-norArg-(18:1(12)), (18:1(5))-norArg-(18:1(7)), (18:1(5))-norArg-(18:1(9)), (18:1(5))-norArg-(18:1(11)), (18:1(5))-norArg-(18:1(12)), (18:1(7))-norArg-(18:1(9)), (18:1(7))-norArg-(18:1(11)), (18:1(7))-norArg-(18:1(12)), (18:1(9))-norArg-(18:1(11)), (18:1(9))-norArg-(18:1(12)), and (18:1(11))-norArg-(18:1(12)).

Examples of an activity-generating delivery molecule include (18:1(3))-norArg-(18:2(9,12)), (18:1(5))-norArg-(18:2(9,12)), (18:1(7))-norArg-(18:2(9,12)), (18:1(9))-norArg-(18:2(9,12)), (18:1(11))-norArg-(18:2(9,12)), and (18:1(12))-norArg-(18:2(9,12)).

Examples of an activity-generating delivery molecule include (18:2(9,12))-norArg-(18:1(3)), (18:2(9,12))-norArg-(18:1(5)), (18:2(9,12))-norArg-(18:1(7)), (18:2(9,12))-norArg-(18:1(9)), (18:2(9,12))-norArg-(18:1(11)), and (18:2(9,12))-norArg-(18:1(12)).

Examples of an activity-generating delivery molecule include (18:2(9,12))-norArg-(18:2(9,12)).

Any of the foregoing activity-generating delivery molecules wherein Xaa is D- or L-norarginine can have a nitrogen atom of the guanidino group of the norarginine quaternized by hydrogen to form ═NH₂⁺, or by one or more methyl, ethyl, propyl or butyl groups to form ═NHR^{|}, or ═NR₉^{|}, which are cationic forms and includes any tautomeric forms.

### His Activity-Generating Delivery Molecules

Examples of an activity-generating delivery molecule of this invention include R³—(C═O)-Xaa-NH—R⁴ wherein R³ and R⁴ are as defined above, and Xaa is D- or L-histidine.

Examples of an activity-generating delivery molecule of this invention include R³—(C═O)-His-NH—R⁴ where His is D- or L-histidine, and R³ and R⁴ are substituted or unsubstituted C(14-24)alkenyl, and salts thereof.

Examples of an activity-generating delivery molecule include R³—(C═O)-His-NH—R⁴ where His is D- or L-histidine, R³—(C═O)— is (18:1)oleoyl, and —NH—R⁴ is (18:1)alkenylamino as defined above.

Examples of an activity-generating delivery molecule include

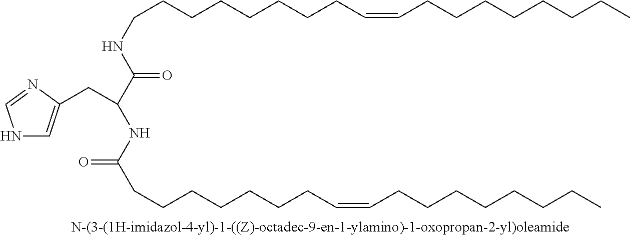 [SMILES: CCCCCCCC/C=C\CCCCCCCCNC(=O)C(Cc1c[nH]cn1)NC(=O)CCCCCCC/C=C\CCCCCCCC]

Examples of an activity-generating delivery molecule include R³—(C═O)-His-NH—R⁴ where His is D- or L-histidine, R³—(C═O)— is (18:1)oleoyl, and —NH—R⁴ is (18:2)alkenylamino as defined above.

Examples of an activity-generating delivery molecule include

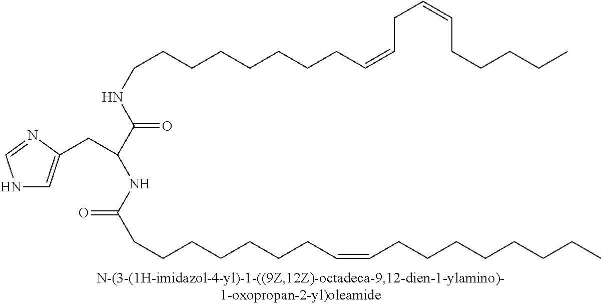 [SMILES: CCCCC/C=C\C/C=C\CCCCCCCCNC(=O)C(Cc1c[nH]cn1)NC(=O)CCCCCCC/C=C\CCCCCCCC]

Examples of an activity-generating delivery molecule include R³—(C═O)-His-NH—R⁴ where His is D- or L-histidine, R³—(C═O)— is (18:2)oleoyl, and —NH—R⁴ is (18:1)alkenylamino as defined above.

Examples of an activity-generating delivery molecule include

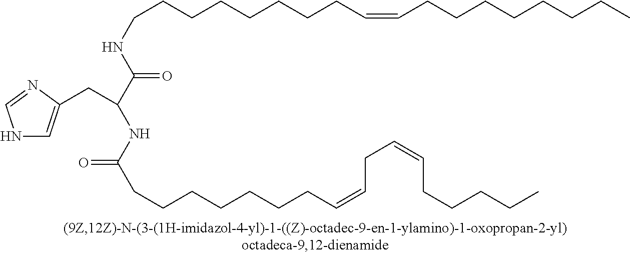 [SMILES: CCCCC/C=C\C/C=C\CCCCCCCC(=O)NC(Cc1c[nH]cn1)C(=O)NCCCCCCCC/C=C\CCCCCCCC]

Examples of an activity-generating delivery molecule include R³—(C═O)-His-NH—R⁴ where His is D- or L-histidine, R³—(C═O)— is (18:2)oleoyl, and —NH—R⁴ is (18:2)alkenylamino.

Examples of an activity-generating delivery molecule include

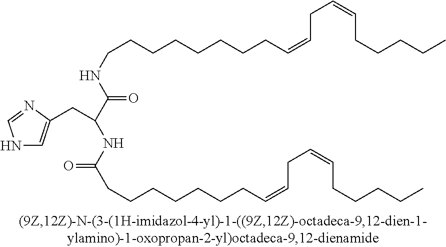 [SMILES: CCCCC/C=C\C/C=C\CCCCCCCCNC(=O)C(Cc1c[nH]cn1)NC(=O)CCCCCCC/C=C\C/C=C\CCCCC]

Examples of an activity-generating delivery molecule include (18:1(3))-His-(18:1(3)), (18:1(5))-His-(18:1 (5)), (18:1(7))-His-(18:1(7)), (18:1(9))-His-(18:1(9)), (18:1(11))-His-(18:1(11)), (18:1(12))-His-(18:1(12)), (18:1(3))-His-(18:1(5)), (18:1(3))-His-(18:1(7)), (18:1(3))-His-(18:1(9)), (18:1(3))-His-(18:1(11)), (18:1(3))-His-(18:1(12)), (18:1(5))-His-(18:1(7)), (18:1(5))-His-(18:1(9)), (18:1(5))-His-(18:1(11)), (18:1(5))-His-(18:1(12)), (18:1(7))-His-(18:1(9)), (18:1(7))-His-(18:1(11)), (18:1(7))-His-(18:1(12)), (18:1(9))-His-(18:1(11)), (18:1(9))-His-(18:1(12)), and (18:1(11))-His-(18:1(12)).

Examples of an activity-generating delivery molecule include (18:1(3))-His-(18:2(9,12)), (18:1(5))-His-(18:2(9,12)), (18:1(7))-His-(18:2(9,12)), (18:1(9))-His-(18:2(9,12)), (18:1(11))-His-(18:2(9,12)), and (18:1(12))-His-(18:2(9,12)).

Examples of an activity-generating delivery molecule include (18:2(9,12))-His-(18:1(3)), (18:2(9,12))-His-(18:1(5)), (18:2(9,12))-His-(18:1(7)), (18:2(9,12))-His-(18:1(9)), (18:2(9,12))-His-(18:1(11)), and (18:2(9,12))-His-(18:1(12)).

Examples of an activity-generating delivery molecule include (18:2(9,12))-His-(18:2(9,12)).

Any of the foregoing activity-generating delivery molecules wherein Xaa is D- or L-histidine can have the hydrogen atom of the side chain of the histidine substituted by a methyl, ethyl, propyl or butyl group to form a side chain N-methyl histidine derivative.

Any of the foregoing activity-generating delivery molecules wherein Xaa is D- or L-histidine can have a nitrogen atom of the side chain of the histidine quaternized by hydrogen to form

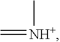

or by a methyl, ethyl, or propyl group to form

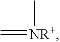

which are or cationic forms and include any tautomeric forms.

### Pro Activity-Generating Delivery Molecules

Examples of an activity-generating delivery molecule of this invention include R³—(C═O)-Xaa-NH—R⁴ wherein R³ and R⁴ are as defined above, and Xaa is D- or L-proline.

Examples of an activity-generating delivery molecule of this invention include R³—(C═O)-Pro-NH—R⁴ where Pro is D- or L-proline, and R³ and R⁴ are substituted or unsubstituted C(14-24)alkenyl, and salts thereof.

Examples of an activity-generating delivery molecule include R³—(C═O)-Pro-NH—R⁴ where Pro is D- or L-proline, R³—(C═O)— is (18:1)oleoyl, and —NH—R⁴ is (18:1)alkenylamino as defined above.

Examples of an activity-generating delivery molecule include

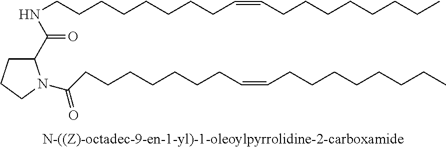 [SMILES: CCCCCCCC/C=C\CCCCCCCCNC(=O)C1CCCN1C(=O)CCCCCCC/C=C\CCCCCCCC]

Examples of an activity-generating delivery molecule include R³—(C═O)-Pro-NH—R⁴ where Pro is D- or L-proline, R³—(C═O)— is (18:1)oleoyl, and —NH—R⁴ is (18:2)1akenylamino.

Examples of an activity-generating delivery molecule include

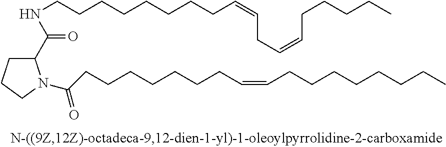 [SMILES: CCCCC/C=C\C/C=C\CCCCCCCCNC(=O)C1CCCN1C(=O)CCCCCCC/C=C\CCCCCCCC]

Examples of an activity-generating delivery molecule include R³—(C═O)-Pro-NH—R⁴ where Pro is D- or L-proline, R³—(C═O)— is (18:2)oleoyl, and —NH—R⁴ is (18:1)alkenylamino.

Examples of an activity-generating delivery molecule include

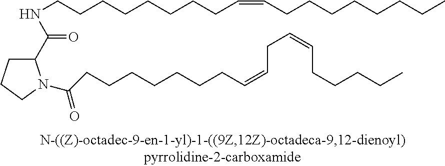 [SMILES: CCCCC/C=C\C/C=C\CCCCCCCC(=O)N1CCCC1C(=O)NCCCCCCCC/C=C\CCCCCCCC]

Examples of an activity-generating delivery molecule include R³—(C═O)-Pro-NH—R⁴ where Pro is D- or L-proline, R³—(C═O)— is (18:2)olcoyl, and —NH—R⁴ is (18:2)alkenylamino.

Examples of an activity-generating delivery molecule include

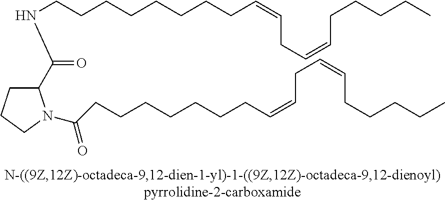 [SMILES: CCCCC/C=C\C/C=C\CCCCCCCCNC(=O)C1CCCN1C(=O)CCCCCCC/C=C\C/C=C\CCCCC]

Examples of an activity-generating delivery molecule include (18:1(3))-Pro-(18:1(3)), (18:1(5))-Pro-(18:1(5)), (18:1(7))-Pro-(18:1(7)), (18:1(9))-Pro-(18:1(9)), (18:1(11))-Pro-(18:1(11)), (18:1(12))-Pro-(18:1(12)), (18:1(3))-Pro-(18:1(5)), (18:1(3))-Pro-(18:1(7)), (18:1(3))-Pro-(18:1(9)), (18:1(3))-Pro-(18:1(11)), (18:1(3))-Pro-(18:1(12)), (18:1(5))-Pro-(18:1(7)), (18:1(5))-Pro-(18:1(9)), (18:1(5))-Pro-(18:1(11)), (18:1(5))-Pro-(18:1(12)), (18:1(7))-Pro-(18:1(9)), (18:1(7))-Pro-(18:1(11)), (18:1(7))-Pro-(18:1(12)), (18:1(9))-Pro-(18:1(11)), (18:1(9))-Pro-(18:1(12)), and (18:1(11))-Pro-(18:1(12)).

Examples of an activity-generating delivery molecule include (18:1(3))-Pro-(18:2(9,12)), (18:1(5))-Pro-(18:2(9,12)), (18:1(7))-Pro-(18:2(9,12)), (18:1(9))-Pro-(18:2(9,12)), (18:1(11))-Pro-(18:2(9,12)), and (18:1(12))-Pro-(18:2(9,12)).

Examples of an activity-generating delivery molecule include (18:2(9,12))-Pro-(18:1(3)), (18:2(9,12))-Pro-(18:1(5)), (18:2(9,12))-Pro-(18:1(7)), (18:2(9,12))-Pro-(18:1(9)), (18:2(9,12))-Pro-(18:1(11)), and (18:2(9,12))-Pro-(18:1(12)).

Examples of an activity-generating delivery molecule include (18:2(9,12))-Pro-(18:2(9,12)).

Any of the foregoing activity-generating delivery molecules wherein Xaa is D- or L-proline can have the side chain of the proline substituted by an amino group to form a 4-aminoproline, or Pro(4-amino), as shown in the following figure:

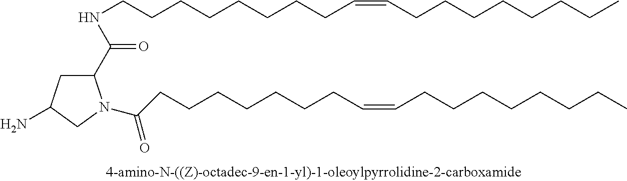 [SMILES: CCCCCCCC/C=C\CCCCCCCCNC(=O)C1CC(N)CN1C(=O)CCCCCCC/C=C\CCCCCCCC]

The foregoing activity-generating delivery molecules wherein Xaa is D- or L-aminoproline can have the nitrogen atom of the amino group of the aminoproline quaternized by hydrogen to form —NH₃⁺, or by one or more methyl, ethyl, propyl or butyl groups (“R” groups) to form —NH₂R⁺, —NHR₂⁺, or —NR₃⁺, which are cationic forms and includes any tautomeric forms.

### Methods for Synthesizing Activity-Generating Compounds and Formulations Thereof

An activity-generating delivery molecule of this disclosure can be synthesized by methods known in the art.

Methods to prepare various organic groups and protective groups are known in the art and their use and modification is generally within the ability of one of skill in the art. See, e.g., Stanley R. Sandler and Wolf Karo, Organic Functional Group Preparations (1989); Greg T. Hermanson, Bioconjugate Techniques (1996); Leroy G. Wade, Compendium Of Organic Synthetic Methods (1980); examples of protective groups are found in T. W. Greene and P. G. M. Wuts, Protective Groups In Organic Synthesis (3rd ed. 1991).

Example methods and processes for making a nanoparticle-containing composition containing an active agent are described in US 2010-0112042 A1 which is incorporated by reference herein in its entirety.

### Nucleic Acid Agents

In certain aspects, this invention provides molecules and methods for generating activity of a nucleic acid agent in a cell or subject. In general, nucleic acids are stable for only limited times when introduced into cells or blood. However, nucleic acid-based agents can be stabilized in compositions and formulations which may then be administered and dispersed for cellular delivery.

Examples of nucleic acid agents include any nucleic acid-containing moieties such as gene-silencing agents, gene-regulating agents, antisense agents, peptide nucleic acid agents, ribozyme agents, RNA agents, and DNA agents.

Examples of an active nucleic acid agent of this disclosure include a UsiRNA. Further examples of nucleic acid agents include two- or three-stranded RNA structures, RNA peptide conjugates, condensed RNA nanoparticles, dicer substrate RNAs, dsRNAs, siRNAs, microRNAs, hairpin RNAs, and other active RNA forms.

The active agent of this disclosure may be a peptide condensate of an active RNA agent. For example, nanoparticles formed by condensing an active RNA agent with a peptide or other biomolecule, condensates of an RNA with a polymeric species, can be loaded as cargo into a nanoparticle composition of this disclosure. The nanoparticles may be crosslinked.

Examples of an active agent of this disclosure include UsiRNAs. A UsiRNA is a UNA-containing siRNA, where a UNA is an “unlocked nucleobase analog.” Examples of a nucleic acid agent of this disclosure may contain one or more acyclic monomers described in PCT International Application Publication No. WO2008/147824.

In addition, as used herein, the terms “dsRNA,” “RNAi-inducing agent,” and “RNAi-agent” are meant to be synonymous with other terms used to describe nucleic acid molecules that are capable of mediating sequence specific RNAi including meroduplex RNA (mdRNA), nicked dsRNA (ndsRNA), gapped dsRNA (gdsRNA), short interfering nucleic acid (siRNA), siRNA, microRNA (miRNA), single strand RNA, short hairpin RNA (shRNA), short interfering oligonucleotide, short interfering substituted oligonucleotide, short interfering modified oligonucleotide, chemically-modified dsRNA, and post-transcriptional gene silencing RNA (ptgsRNA), as well as precursors of any of the above.

The term “large double-stranded (ds) RNA” refers to any double-stranded RNA longer than about 40 base pairs (bp) to about 100 bp or more, particularly up to about 300 bp to about 500 bp. The sequence of a large dsRNA may represent a segment of an mRNA or an entire mRNA. A double-stranded structure may be formed by self-complementary nucleic acid molecule or by annealing of two or more distinct complementary nucleic acid molecule strands.

### Additional Active Agents

The compounds and compositions of this disclosure may be used for delivery of any physiologically or biologically active agent, as well as any combination of active agents, as described above or known in the art. The active agent may be present in the compositions and uses of this disclosure in an amount sufficient to provide the desired physiological or ameliorative effect.

The compounds and compositions of this disclosure are directed toward enhancing delivery of a range of drug agents and biologically active agents in mammalian subjects including small molecule compounds and drugs, peptides, proteins, antibodies, monoclonal antibodies, antibody-based drugs, and vaccine agents.

Examples of an active agent include a peptide, a protein, a protease, an antibody, a monoclonal antibody, an antibody-based drug, a vaccine agent, or a small molecule drug.

Examples of active agents include a peptide, a protein, a nucleic acid, a double-stranded RNA, a hematopoietic, an antiinfective; an antidementia; an antiviral, an antitumoral, an antipyretic, an analgesic, an anti-inflammatory, an antiulcerative, an antiallergenic, an antidepressant, a psychotropic, a cardiotonic, an antiarrythmic, a vasodilator, an antihypertensive, a hypotensive diuretic, an antidiabetic, an anticoagulant, a cholesterol-lowering agent, a therapeutic for osteoporosis, a hormone, an antibiotic, a vaccine, a cytokine, a hormone, a growth factor, a cardiovascular factor, a cell adhesion factor, a central or peripheral nervous system factor, a humoral electrolyte factor, a hemal organic substance, a bone growth factor, a gastrointestinal factor, a kidney factor, a connective tissue factor, a sense organ factor, an immune system factor, a respiratory system factor, a genital organ factor, an androgen, an estrogen, a prostaglandin, a somatotropin, a gonadotropin, an interleukin, a steroid, a bacterial toxoid, an antibody, a monoclonal antibody, a polyclonal antibody, a humanized antibody, an antibody fragment, and an immunoglobin.

Examples of active agents include erythropoietin, granulocyte-colony stimulating factor, insulin, Factor VII, Factor VIII, Factor IX, interferon, heparin, hirugen, hirulos, and hirudine.

Examples of active agents include morphine, hydromorphone, oxymorphone, lovorphanol, levallorphan, codeine, nalmefene, nalorphine, nalozone, naltrexone, buprenorphine, butorphanol, or nalbufine, cortisone, hydrocortisone, fludrocortisone, prednisone, prednisolone, methylprednisolone, triamcinolone, dexamethoas one, betamethoasone, paramethosone, fluocinolone, colchicine, acetaminophen, a non-steroidal anti-inflammatory agent NSAID, acyclovir, ribavarin, trifluorothyridine, Ara-A Arabinofuranosyladenine, acylguanosine, nordeoxyguanosine, azidothymidine, dideoxyadenosine, dideoxycytidine, spironolactone, testosterone, estradiol, progestin, gonadotrophin, estrogen, progesterone, papaverine, nitroglycerin, a vasoactive intestinal peptide, calcitonin gene-related peptide, cyproheptadine, doxepin, imipramine, cimetidine, dextromethorphan, clozaril, superoxide dismutase, neuroenkephalinase, amphotericin B, griseofulvin, miconazole, ketoconazole, tioconazol, itraconazole, fluconazole, cephalosporin, tetracycline, aminoglucoside, erythromicin, gentamicin, polymyxin B, 5-fluorouracil, bleomycin, methotrexate, hydroxyurea, dideoxyinosine, floxuridine, 6-mercaptopurine, doxorubicin, daunorubicin, I-darubicin, taxol, paclitaxel, tocopherol, quinidine, prazosin, verapamil, nifedipine, diltiazem, tissue plasminogen activator TPA, epidermal growth factor EGF, fibroblast growth factor FGF-acidic or basic, platelet derived growth factor PDGF, transforming growth factor TGF-alpha or beta, vasoactive intestinal peptide, tumor necrosis factor TNF, hypothalmic releasing factor, prolactin, thyroid stimulating hormone TSH, adrenocorticotropic hormone ACTH, parathyroid hormone PTH, follicle stimulating hormone FSF, luteinizing hormone releasing hormone LHRH, endorphin, glucagon, calcitonin, oxytocin, aldoetecone, enkaphalin, somatostin, somatotropin, somatomedin, alpha-melanocyte stimulating hormone, lidocaine, sufentainil, terbutaline, droperidol, scopolamine, gonadorelin, ciclopirox, buspirone, cromolyn sodium, midazolam, cyclosporin, lisinopril, captopril, delapril, ranitidine, famotidine, superoxide dismutase, asparaginase, arginase, arginine deaminease, adenosine deaminase ribonuclease, trypsin, chemotrypsin, papain, bombesin, substance P, vasopressin, alpha-globulins, transferrin, fibrinogen, beta-lipoprotein, beta-globulin, prothrombin, ccruloplasmin, alpha2-glycoprotein, alpha2-globulin, fctuin, alpha1-lipoprotein, alpha1-globulin, albumin, and prealbumin.

Examples of active agents include opioids or opioid antagonists, such as morphine, hydromorphone, oxymorphone, lovorphanol, levallorphan, codeine, nalmefene, nalorphine, nalozone, naltrexone, buprenorphine, butorphanol, and nalbufine; corticosterones, such as cortisone, hydrocortisone, fludrocortisone, prednisone, prednisolone, methylprednisolone, triamcinolone, dexamethoasone, betamethoasone, paramethosone, and fluocinolone; other anti-inflammatories, such as colchicine, ibuprofen, indomethacin, and piroxicam; anti-viral agents such as acyclovir, ribavarin, trifluorothyridine, Ara-A (Arabinofuranosyladenine), acylguanosine, nordeoxyguanosine, azidothymidine, dideoxyadenosine, and dideoxycytidine; antiandrogens such as spironolactone; androgens, such as testosterone; estrogens, such as estradiol; progestins; muscle relaxants, such as papaverine; vasodilators, such as nitroglycerin, vasoactive intestinal peptide and calcitonin related gene peptide; antihistamines, such as cyproheptadine; agents with histamine receptor site blocking activity, such as doxepin, imipramine, and cimetidine; antitussives, such as dextromethorphan; neuroleptics such as clozaril; antiarrhythmics; antiepileptics; enzymes, such as superoxide dismutase and neuroenkephalinase; anti-fungal agents, such as amphotericin B, griseofulvin, miconazole, ketoconazole, tioconazol, itraconazole, and fluconazole; antibacterials, such as penicillins, cephalosporins, tetracyclines, aminoglucosides, erythromicin, gentamicins, polymyxin B; anti-cancer agents, such as 5-fluorouracil, bleomycin, methotrexate, and hydroxyurea, dideoxyinosine, floxuridine, 6-mercaptopurine, doxorubicin, daunorubicin, I-darubicin, taxol, and paclitaxel; antioxidants, such as tocopherols, retinoids, carotenoids, ubiquinones, metal chelators, and phytic acid; antiarrhythmic agents, such as quinidine; antihypertensive agents such as prazosin, verapamil, nifedipine, and diltiazem; analgesics such as acetaminophen and aspirin; monoclonal and polyclonal antibodies, including humanized antibodies, and antibody fragments; anti-sense oligonucleotides; and RNA, regulatory RNA, interfering RNA, DNA, and viral vectors comprising genes encoding therapeutic peptides and proteins.

Use of Activity-Generating Delivery Molecules in Formulations with an RNA Agent

Activity-generating delivery molecules of this disclosure may be used for delivery of drug agents or biologically active agents to a variety of cells, tissues or organs in vivo. Modalities for delivering an agent in vivo include topical, enteral, and parenteral routes. Examples of modalities for delivering an agent in vivo include inhalation of particles or droplets, delivery of nasal or nasal-pharngyl drops, particles, or suspensions, transdermal and transmucosal routes, as well as injection or infusion by intramuscular, subcutaneous, intravenous, intraarterial, intracardiac, intrathecal, intraosseus, intraperitoneal, and epidural routes. In some embodiments, an agent can be administered ex vivo by direct exposure to cells, tissues or organs originating from a mammalian subject.

In some embodiments, this disclosure provides a method for treating a disease or disorder in a mammalian subject. A therapeutically effective amount of a composition of this disclosure containing an active RNA agent and one or more activity-generating delivery molecules, along with other excipients, may be administered to a subject having a disease or disorder associated with expression or overexpression of a gene that can be reduced, decreased, downregulated, or silenced by the composition.

Acceptable solvents, vehicles, or diluents for therapeutic use are well known in the pharmaceutical art, and are described, for example, in Remington's Pharmaceutical Sciences, Mack Publishing Co. (A. R. Gennaro ed. 1985).

A pharmaceutically effective dose that is required to prevent, inhibit the occurrence of, treat, or alleviate a symptom of a disease state includes an amount of from 0.01 mg/kg to 50 mg/kg body weight/day of active nucleic acid should be administered.

This disclosure encompasses methods for treating a disease including cancer, bladder cancer, liver cancer, liver disease, hypercholesterolemia, an inflammatory disease, a metabolic disease, inflammation, arthritis, rheumatoid arthritis, encephalitis, bone fracture, heart disease, viral disease, hepatitis, and influenza.

A drug agent or biologically active agent to be delivered using a composition or formulation of this disclosure may be found in any form including, for example, a pure form, a crystalline form, a solid form, a nanoparticle, a condensed form, a complexed form, or a conjugated form.

This disclosure further provides a range of pharmaceutically acceptable nucleic acid compositions with various activity-generating delivery molecules for therapeutic delivery of a nucleic acid agent or gene-silencing RNA.

In particular, this disclosure provides formulations of activity-generating delivery molecules and methods for in vitro and in vivo delivery of an active RNA agent for decreasing, downregulating, or silencing the translation of a target nucleic acid sequence or expression of a gene. These formulations of activity-generating delivery molecules may be used for prevention or treatment of diseases in a mammal.

In some aspects, this disclosure provides a range of formulations including one or more activity-generating delivery molecules of this disclosure and one or more lipids which may be used for delivery and administration of a nucleic acid agent.

More particularly, a composition of this disclosure may include one or more activity-generating delivery molecules of this invention along with one or more cationic lipids or non-cationic lipids. A composition of this disclosure may include one or more activity-generating delivery molecules of this invention along with one or more cationic lipids and one or more non-cationic lipids.

Cationic lipids may be monocationic or polycationic. Some cationic lipids include neutral lipids and lipids having approximately zero net charge at a particular pH, for example, a zwitterionic lipid. Non-cationic lipids also include anionic lipids.

Examples of neutral lipids include cholesterol, DOPC, DOPE, DDPC, DDPE, DLPC, DLPE, DMPC, DMPE, DPPC, DPPE, DSPC, DSPE, DPhyPE, sphingomylin, ceramides, diacylglycerols, and sphingosine.

Examples of cationic lipids include DOTAP, DC-CHOL, DOTMA, Ethyl PC, DDAB, and DODAP.

Examples of anionic lipids include CHEMS, DOPS, POPS, DLPS, DMPS, DPPS, DOPI, POPI, DMPI, or DPPI.

Non-cationic lipids include neutral, zwitterionic, and anionic lipids. Thus, a non-cationic zwitterionic lipid may contain a cationic head group.

Activity-generating delivery molecules of this disclosure may be admixed with, or attached to various targeting ligands or agents to deliver an active agent to a cell, tissue, organ or region of an organism. Examples of targeting agents include antibodies, ligands for receptors, peptides, proteins, lectins, (poly)saccharides, galactose, mannose, cyclodextrins, nucleic acids, DNA, RNA, aptamers, and polyamino acids.

Methods for making a nucleic acid composition of an activity-generating delivery molecule of this invention include ethanol injection methods and extrusion methods using a Northern Lipids Lipex Extruder system with stacked polycarbonate membrane filters of defined pore size. Sonication using probe tip and bath sonicators can be employed to produce particles of uniform size. Homogenous and monodisperse particle sizes can be obtained without the addition of the nucleic acid component. For in vitro transfection compositions, the nucleic acid component can be added after the transfection agent is made and stabilized by buffer components. For in vivo delivery compositions, the nucleic acid component is part of the formulation.

A formulation containing an activity-generating delivery molecule of this disclosure may be administered by various routes, for example, to effect systemic delivery via intravenous, parenteral, or intraperitoneal routes. In some embodiments, an agent may be delivered intracellularly, for example, in cells of a target tissue such as lung or liver, or in inflamed tissues. Included within this disclosure are compositions and methods for delivery of an agent by removing cells of a subject, delivering an agent to the removed cells, and reintroducing the cells into a subject. In some embodiments, this disclosure provides a method for delivery of an agent in vivo. A composition may be administered intravenously, subcutaneously, or intraperitoneally to a subject. In some embodiments, the disclosure provides methods for in vivo delivery of an agent to the lung of a mammalian subject.

A formulation containing an activity-generating delivery molecule of this disclosure may be used in pharmaceutical compositions of an active agent in vivo. Administration of the active agent composition of this disclosure to a subject may be parenteral, oral, by inhalation, topical, mucosal, rectal, or buccal routes. Parenteral use includes subcutaneous, intracutaneous, intravenous, intramuscular, intraarticular, intrasynovial, intrastemal, intrathecal, intralesional, and intracranial injection or infusion techniques.

An effective amount of an active agent composition of this disclosure for treating a particular disease is generally an amount sufficient to ameliorate or reduce a symptom of the disease. The composition may be administered as a single dosage, or may be administered by repeated dosing.

### Chemical Definitions

It will be understood that a drawing of a molecule in this disclosure that has an explicit charge shall include a counterion which is pharmaceutically-acceptable, whether or not the counterion is expressly included in the drawing.

As used herein, the term “homo,” when referring to an amino acid, means that an additional carbon is added to the side chain, while the term “nor,” when referring to an amino acid, means that a carbon is subtracted from the side chain. Thus, homolysine refers to side chain: (CH₂)₅NH₂.

In general, as used herein, general chemical terms refer to all groups of a specified type, including groups having any number and type of atoms, unless otherwise specified. For example “alkenyl” refers broadly to alkyls having 2 to 24 carbon atoms, as defined below, while (C18:1)alkenyl refers to alkenyls having 18 carbon atoms and one double bond.

The term “alkyl” as used herein refers to a saturated, branched or unbranched, substituted or unsubstituted aliphatic group containing from 1-24 carbon atoms. This definition applies to the alkyl portion of other groups such as, for example, alkoxy, alkanoyl, aralkyl, and other groups defined below. The term “cycloalkyl” as used herein refers to a saturated, substituted or unsubstituted cyclic alkyl ring containing from 3 to 12 carbon atoms.

Examples of substituents for an alkyl group include alkyl, alkenyl, and aryl substituents including methyl, ethyl, n-propyl, i-propyl, n-butyl, i-butyl, t-butyl, sec-butyl, vinyl or ethenyl, allyl or 2-propenyl, 1-propenyl, isopropenyl or 1-methylvinyl, 2-butenyl, 1,3-butadienyl, 2-pentenyl, phenyl, and naphthyl.

Examples of alkyl groups include methyl, ethyl, n-propyl, i-propyl, n-butyl, butyl, t-butyl, and sec-butyl. Examples of cycloalkyls include cyclopropane, cyclobutane, cyclopentane, cyclohexane, and cycloheptane.

Examples of substituents for an alkyl group include alkyl, alkenyl, and aryl substituents including methyl, ethyl, n-propyl, i-propyl, n-butyl, i-butyl, t-butyl, sec-butyl, vinyl or ethenyl, allyl or 2-propenyl, 1-propenyl, isopropenyl or 1-methylvinyl, 2-butenyl, 1,3-butadienyl, 2-pentenyl, phenyl, and naphthyl.

The term “alkenyl” as used herein refers to an unsaturated, branched or unbranched, substituted or unsubstituted alkyl or cycloalkyl having 2 to 24 carbon atoms and at least one carbon-carbon double bond. The term “alkynyl” as used herein refers to an unsaturated, branched or unbranched, substituted or unsubstituted alkyl or cycloalkyl having 2 to 24 carbon atoms and at least one carbon-carbon triple bond.

Examples of alkenyl groups include vinyl or ethenyl, allyl or 2-propenyl, 1-propenyl, isopropenyl or 1-methylvinyl, 2-butenyl, 1,3-butadienyl, and 2-pentenyl.

Examples of substituents for an alkenyl group include alkyl, alkenyl, and aryl substituents including methyl, ethyl, n-propyl, i-propyl, n-butyl, i-butyl, t-butyl, sec-butyl, vinyl or ethenyl, allyl or 2-propenyl, 1-propenyl, isopropenyl or 1-methylvinyl, 2-butenyl, 1,3-butadienyl, 2-pentenyl, phenyl, and naphthyl.

The term “alkoxy” as used herein refers to an alkyl, cycloalkyl, alkenyl, or alkynyl group covalently bonded to an oxygen atom. The term “alkanoyl” as used herein refers to —C(═O)-alkyl, which may alternatively be referred to as “acyl.” The term “alkanoyloxy” as used herein refers to —O—C(═O)-alkyl groups. The term “alkylamino” as used herein refers to the group —NRR′, where R and R′ are each either hydrogen or alkyl, and at least one of R and R′ is alkyl. Alkylamino includes groups such as piperidino, wherein R and R′ form a ring. The term “alkylaminoalkyl” refers to -alkyl-NRR′.

The term “aryl” as used herein refers to any stable monocyclic, bicyclic, or polycyclic carbon ring system of from 4 to 12 atoms in each ring, wherein at least one ring is aromatic. Some examples of an aryl include phenyl, naphthyl, tetrahydro-naphthyl, indanyl, and biphenyl. Where an aryl substituent is bicyclic and one ring is non-aromatic, it is understood that attachment is to the aromatic ring.

An aryl may be substituted or unsubstituted. Examples of substituents for an aryl group include alkyl, alkenyl, and aryl substituents including methyl, ethyl, n-propyl, i-propyl, n-butyl, i-butyl, t-butyl, sec-butyl, vinyl or ethenyl, allyl or 2-propenyl, 1-propenyl, isopropenyl or 1-methylvinyl, 2-butenyl, 1,3-butadienyl, 2-pentenyl, phenyl, and naphthyl.

The term “heteroaryl” as used herein refers to any stable monocyclic, bicyclic, or polycyclic carbon ring system of from 4 to 12 atoms in each ring, wherein at least one ring is aromatic and contains from 1 to 4 heteroatoms selected from oxygen, nitrogen and sulfur. Some examples of a heteroaryl include acridinyl, quinoxalinyl, pyrazolyl or pyrazolidinyl, indolyl, benzotriazolyl, furanyl, thienyl, benzothienyl, benzofuranyl, quinolinyl, isoquinolinyl, oxazolyl, oxadiazolyl, is oxazolyl, thiazolyl, is othiazolyl, thiadiazolyl, imidazolyl, pyridazolyl, pyrazolyl, pyrazinyl, pyridazinyl, pyridinyl or pyridyl, pyrimidinyl, pyrrolyl, and tetrahydroquinolinyl. A heteroaryl includes the N-oxide derivative of a nitrogen-containing heteroaryl.

The term “heterocycle” or “heterocyclyl” as used herein refers to an aromatic or nonaromatic ring system of from five to twenty-two atoms, wherein from 1 to 4 of the ring atoms are heteroatoms selected from oxygen, nitrogen, and sulfur. Thus, a heterocycle may be a heteroaryl or a dihydro or tetrathydro version thereof.

Examples of a heterocycle group or moiety include a monocyclic non-aromatic, saturated or unsaturated C₅-C₁₀ carbocyclic ring in which one or more, for example 1, 2 or 3, of the carbon atoms are replaced with a moiety selected from N, O, S, S(O) and S(O)₂. Suitable heterocyclyl groups and moieties include pyrazolidinyl, piperidyl, piperazinyl, thiomorpholinyl, S-oxo-thiomorpholinyl, S,S-dioxo-thiomorpholinyl, morpholinyl, pyrrolidinyl, pyrrolinyl, imidazolidinyl, imidazolinyl, 1,3-dioxolanyl, 1,4-dioxolyl and pyrazolinyl groups and moieties.

The term “aroyl” as used herein refers to an aryl radical derived from an aromatic carboxylic acid, such as a substituted benzoic acid. The term “aralkyl” as used herein refers to an aryl group bonded to an alkyl group, for example, a benzyl group.

The term “carboxyl” as used herein represents a group of the formula —C(═O)OH or —C(═O)O⁻. The terms “carbonyl” and “acyl” as used herein refer to a group in which an oxygen atom is double-bonded to a carbon atom >C═O. The term “hydroxyl” as used herein refers to —OH or The term “nitrile” or “cyano” as used herein refers to —CN. The term “halogen” or “halo” refers to fluoro (—F), chloro (—Cl), bromo (—Br), and iodo (—I).

The term “substituted” as used herein refers to an atom having one or more substitutions or substituents which can be the same or different and may include a hydrogen substituent. Thus, the terms alkyl, cycloalkyl, alkenyl, alkynyl, alkoxy, alkanoyl, alkanoyloxy, alkylamino, alkylaminoalkyl, aryl, heteroaryl, heterocycle, aroyl, and aralkyl as used herein refer to groups which include substituted variations. Substituted variations include linear, branched, and cyclic variations, and groups having a substituent or substituents replacing one or more hydrogens attached to any carbon atom of the group. Substituents that may be attached to a carbon atom of the group include alkyl, cycloalkyl, alkenyl, alkynyl, alkoxy, alkanoyl, alkanoyloxy, alkylamino, alkylaminoalkyl, aryl, heteroaryl, heterocycle, aroyl, aralkyl, acyl, hydroxyl, cyano, halo, haloalkyl, amino, aminoacyl, alkylaminoacyl, acyloxy, aryloxy, aryloxyalkyl, mercapto, nitro, carbamyl, carbamoyl, and heterocycle. For example, the term ethyl includes without limitation —CH₂CH₃, —CHFCH₃, —CF₂CH₃, —CHFCH₂F, —CHFCHF₂, —CHFCF₃, —CF₂CH₂F, —CF₂CHF₂, —CF₂CF₃, and other variations as described above. In general, substituents may be further substituted with any atom or group of atoms.

Examples of substituents include alkyl, alkenyl, and aryl substituents including methyl, ethyl, n-propyl, i-propyl, n-butyl, i-butyl, t-butyl, sec-butyl, vinyl or ethenyl, allyl or 2-propenyl, 1-propenyl, isopropenyl or 1-mcthylvinyl, 2-butenyl, 1,3-butadienyl, 2-pentenyl, phenyl, and naphthyl.

A pharmaceutically acceptable salt of an activity-generating delivery molecule of this disclosure which is sufficiently basic may be an acid-addition salt with, for example, an inorganic or organic acid such as hydrochloric, hydrobromic, sulfuric, nitric, phosphoric, diphosphoric, chlorosulfonic, trifluoroacetic, citric, maleic, acetic, propionic, oxalic, malic, maleic, malonic, fumaric, ascorbic, succinic, benzoic, or tartaric acids, and alkane- or arenesulfonic acids such as methanesulfonic, ethanesulfonic, benzenesulfonic, chlorobenzenesulfonic, toluenesulfonic, naphthalenesulfonic, naphthalenedisulfonic, and camphorsulfonic acids.

A pharmaceutically acceptable salt of an activity-generating delivery molecule of this disclosure which is sufficiently acidic may be an alkali metal salt, for example, a sodium or potassium salt, or an alkaline earth metal salt, for example, a calcium or magnesium salt, or a zinc or manganese salt, or an ammonium salt or a salt with an organic base which provides a physiologically-acceptable cation, for example, a salt with methylamine, dimethylamine, trimethylamine, triethylamine, ethanolamine, diethanolamine, triethanolamine, ethylenediamine, tromethamine, N-methylglucamine, piperidine, morpholine or tris-(2-hydroxyethyl)amine, and including salts of amino acids such as arginate, and salts of organic acids such as glucuronic or galactunoric acids. See, for example, Berge et al., J. Pharm. Sci. 66:1-19, 1977.

Some compounds of this disclosure may contain both basic and acidic functionalities that may allow the compounds to be made into either a base or acid addition salt.

Some compounds, peptides and/or protein compositions of this disclosure may have one or more chiral centers and/or geometric isomeric centers (E- and Z-isomers), and it is to be understood that the disclosure encompasses all such optical isomers, diastereoisomers, geometric isomers, and mixtures thereof; even where only one isomer appears in a drawing.

This disclosure encompasses any and all tautomeric, solvated or unsolvated, hydrated or unhydrated forms, as well as any atom isotope forms of the compounds, peptides and/or protein compositions disclosed herein.

Compounds of this disclosure containing one or more chiral centers may be used in enantiomerically or diastereoisomerically pure form, or in the form of a mixture of isomers. For the avoidance of doubt, compounds of this disclosure can, if desired, be used in the form of solvates. Further, for the avoidance of doubt, the compounds of the invention may be used in any tautomeric form.

### Additional Embodiments

All publications, references, patents, patent publications and patent applications cited herein are each hereby specifically incorporated by reference in entirety.

While this disclosure has been described in relation to certain embodiments, and many details have been set forth for purposes of illustration, it will be apparent to those skilled in the art that this disclosure includes additional embodiments, and that some of the details described herein may be varied considerably without departing from this disclosure. This disclosure includes such additional embodiments, modifications and equivalents. In particular, this disclosure includes any combination of the features, terms, or elements of the various illustrative components and examples.

The use herein of the terms “a,” “an,” “the” and similar terms in describing the disclosure, and in the claims, are to be construed to include both the singular and the plural.

The terms “comprising,” “having,” “including” and “containing” are to be construed as open-ended terms which mean, for example, “including, but not limited to.” Thus, terms such as “comprising,” “having,” “including” and “containing” are to be construed as being inclusive, not exclusive.

Recitation of a range of values herein refers individually to each and any separate value falling within the range as if it were individually recited herein, whether or not some of the values within the range are expressly recited. For example, the range “4 to 12” includes without limitation the values 5, 5.1, 5.35 and any other whole, integer, fractional, or rational value greater than or equal to 4 and less than or equal to 12. Specific values employed herein will be understood as exemplary and not to limit the scope of the disclosure.

Recitation of a range of number of carbon atoms herein refers individually to each and any separate value falling within the range as if it were individually recited herein, whether or not some of the values within the range are expressly recited. For example, the term “C1-24” includes without limitation the species C1, C2, C3, C4, C5, C6, C7, C8, C9, C10, C11, C12, C13, C14, C15, C16, C17, C18, C19, C20, C21, C22, C23, and C24.

Definitions of technical terms provided herein should be construed to include without recitation those meanings associated with these terms known to those skilled in the art, and are not intended to limit the scope of the disclosure. Definitions of technical terms provided herein shall be construed to dominate over alternative definitions in the art or definitions which become incorporated herein by reference to the extent that the alternative definitions conflict with the definition provided herein.

The examples given herein, and the exemplary language used herein are solely for the purpose of illustration, and are not intended to limit the scope of the disclosure.

When a list of examples is given, such as a list of compounds or molecules suitable for this disclosure, it will be apparent to those skilled in the art that mixtures of the listed compounds or molecules are also suitable.

### EXAMPLES

### Example 1

### In Vivo Gene Silencing Activity Generated with Formulations of C18:2-DAP(N,N-diMe)-C18:2

The synthetic activity-generating delivery molecules of this invention advantageously provide gene-silencing activity in vivo with an agent for RNA interference.

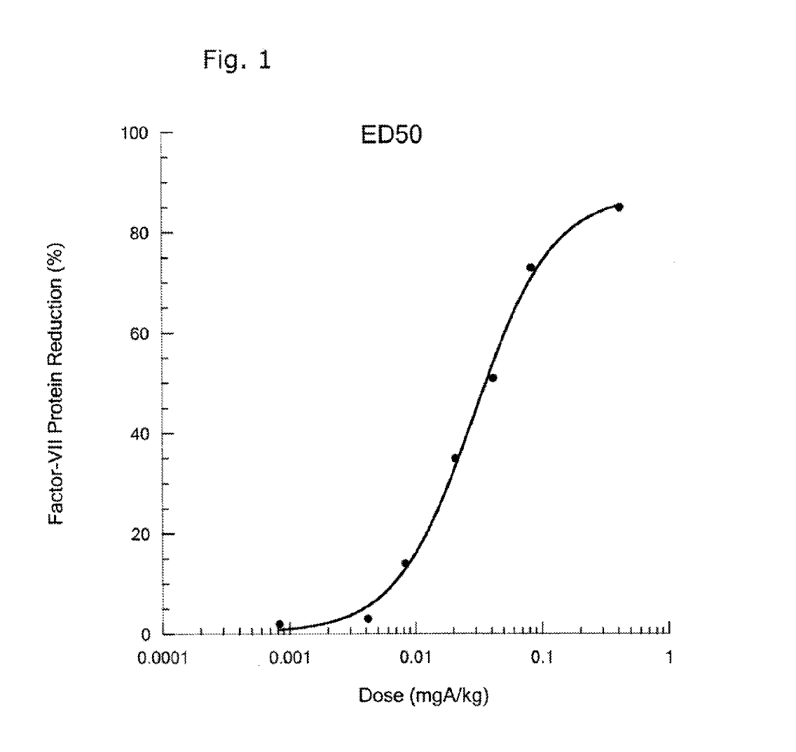

In FIG. 1 is shown a chart of the gene-silencing dose-response in vivo mouse for a UsiRNA against Factor VII administered by tail-vein injection in a formulation including the activity-generating delivery molecule C18:2-DAP(N,N-diMe)-C18:2. The calculated ED50 was 30 μg/kg. The x-axis of FIG. 1 refers to mgA/kg which is by pharmaceutical convention the mg of active UsiRNA per kg body weight. Here it refers to the fraction of UsiRNA that is duplexed, and encapsulated or carried by the activity-generating delivery molecules.

This data was obtained in normal Balb/c mice with dose levels of 0.5, 0.1, 0.05, 0.025, 0.01, 0.005, and 0.001 mg/kg. Animals were each administered a single tail-vein IV injection, and sacrificed at 48 hr post dose for analysis of serum F-VII.

The activity-generating molecule C18:2-DAP(N,N-diMe)-C18:2 was formulated with cholesterol, a second activity-generating molecule C18:2-DAB-C16, and DSPE-PEG2k at concentrations of 51:31:17:1 mole %, respectively.

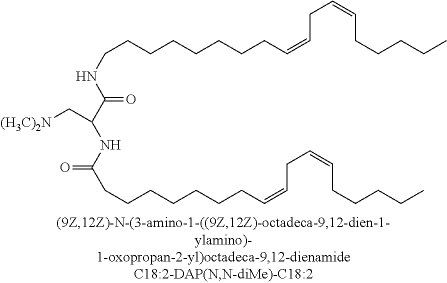

### Example 2

### Preparation of C18:2-DAP(N,N-diMe)-C18:2

### Synthesis of Boc-DAP(N,N-diMe)-OH

A solution of triphenylphosphine in CH₃CN was cooled to 0° C. and diisopropyl azodicarboxylate (DIAD) was added dropwise with a syringe. The resulting pale yellow solution was stirred for 15 minutes at 0° C. (till all solid dissolves), and a solution of Boc-Ser-OH in CH₃CN was slowly added. After completion of the addition the mixture was stirred at 0° C. for 10 minutes, cooling bath was removed and the mixture was slowly war med with stirring. Stirring was continued overnight, solvent removed under reduced pressure and the residue immediately purified by flash chromatography, with hexane/ethyl acetate gradient solvent system. After removing solvents under reduced pressure the purified compound was dissolved in CH₃CN and N,N-dimethyltrimethylsilylamine was added to a solution of lactone. Stirring was continued at r.t for 1.5 and then reaction mixture was concentrated and methanol was added followed by citric acid. After 5 h and resulting solid was filtered off and solvent removed under reduced pressure. The residue was taken between DCM/H2O, aqueous phase back extracted with DCM, concentrated and lyophilized giving off-white solid.

### Synthesis of Linoleyl amine

A mixture of potassium phthalimide (1 eq) and linoleyl methanesulfonate (1 eq) was stirred at 70° C. under nitrogen atmosphere. 100% conversion of starting material was observed by TLC after 4 hrs of stirring. After cooling down to about 50° C. reaction mixture was poured into and water and product was extracted with EtOAc. Combined organic fractions were washed with water, dried over Na₂SO₄, solvent was removed under reduced pressure and the oily-solid residue was treated with hexane. Solid was filtered off, filtrate was evaporated to dryness and crude linoleyl phthalimide was used in the next step. The residue was dissolved in EtOH and hydrazine was added. The mixture was mildly refluxed (85-90° C.) for 2 h. The resulting thick white solid was filtered upon cooling the mixture to about 50° C. and washed with warm ethanol. The filtrate was concentrated almost to dryness and chloroform was added. Resulting white solid was filtered off again and the organic phase was washed twice with water and dried over Na₂CO₃. Solvent was removed to afford crude yellow oil (yield 95%).

### Synthesis of C18:2-DAP(N,N-diMe)-C18:2

Boc-DAP(N,N-diMe)-OH was preactivated with 3-(Diethoxyphosphoryloxy)-1, 2, 3-benzotriazin-4(3H)-one (DEPBT) and 2 eq of DIPEA in THF/DCM solvent mixture for 10 minutes followed by addition of linoleyl amine and subsequent stirring for 30 minutes. Crude compound was purified twice by flash chromatography: 1) normal phase silica gel (DCM/MeOH gradient) and 2) amine capped silica gel (Hexane/AcOEt gradient). The pure monoalkylated intermediate was dissolved in 1M HCl/ethyl acetate solution and the Boc group was removed within one hour followed by removal of the solvent under reduced pressure. The second alkyl chain was attached by preactivating the free carboxyl group of linoleic acid with (1-Ethyl-3-(3-dimethyllaminopropyl)-carbodiimide hydrochloride) (EDC) and N-Hydroxybenzotriazole (HOBt) in a 1:1 mixture of DMF and DCM for 10 minutes followed by addition of monoalkylated diMeDAP dissolved in DCM (pH adjusted to 6 with DIPEA) and subsequent stirring for 30 minutes. Crude compound was purified by flash chromatography (Hexane/AcOEt gradient) and converted to hydrochloride salt by stirring with 1M HCl/AcOEt. Final product was lyophilized.

The pKa for this compound as measured by TNS dye assay was 5.8.

### Example 3

### Preparation of C18:1-DAP(NH₃⁺Cl⁻)—C18:1

C18:1-DAP-C18:1 was synthesized as follows. Fmoc-Nβ-Boc-L-2,3-diaminopropionic acid was dissolved in dichloromethane (DCM), 2eq of diisopropylethyl amine (DIPEA) and the resulting solution was added to 2-chlorotrityl choride resin. After one hour, the resin was washed with DCM and Fmoc group was removed by treatment with 20% piperidine in DMF yielding the free α-amine. Oleic acid was preactivated with 2-(6-Chloro-1H-benzotriazole-1-yl)-1,1,3,3-tetramethylaminium hexafluorophosphate (HCTU) and 2 equivalents of DIPEA and added to the resin and the reaction was deemed complete by negative Kaiser test. The lipidated compound was cleaved from the resin by multiple treatments with 1% trifluoroacetic acid (TFA) in dichloromethane followed by evaporation under reduced pressure yielding free carboxylate intermediate. The second alkyl chain was attached by preactivating the free carboxyl group with (1-Ethyl-3-(3-dimethyllaminopropyl)-carbodiimide hydrochloride) (EDC) and N-Hydroxybenzotriazole (HOBt) in a 1:1 mixture of DMF and DCM for 10 minutes followed by addition of C(18:1)amine in same solvent and subsequent stirring for 30 minutes. Crude compound was purified by flash chromatography (Hexane/AcOEt gradient). The pure dialkylated intermediate was dissolved in 1M HCl/ethyl acetate solution and the Boc group was removed within one hour followed by removal of the solvent under reduced pressure and resulting residue was washed with water and dried.

The pKa's for this compound as measured by TNS dye assay were 6.0 and 9.7.

### Example 4

### Preparation of C18:2-DAA(NH₃

C18:2-DAA-C18:2 was synthesized following methods as for Example 3 with appropriate components.

The pKa for this compound as measured by TNS dye assay was 4.9.

### Example 5

### Preparation of C18:2-DAP(NH₃⁺Cl⁻)—C18:2

C18:2-DAP-C18:2 was synthesized following methods as for Example 3 with appropriate components. The pKa for this compound as measured by TNS dye assay was 7.6.

### Example 6

### Preparation of C18:2-DAP(Me,Me)-C18:1

C18:2-DAP(Me,Me)-C18:1 was synthesized following methods as for Example 3 with appropriate components and the following additional step: To crude hydrochloride of amino acid with 2 aliphatic chains from the previous step dissolved in MeOH, 10 eq of AcOH and 10 eq of 37% formaldehyde were added and reaction solution was heated up to very gentle reflux. Then 10 eq of sodium triacetoxyborohydride were added. The reaction was stirred for 30 min, cooled down to room temperature and worked up by the addition of H₂O and saturated NaHCO3. Product was extracted with DCM and organic layer was dried over MgSO4, filtered and organic solvent was removed under reduced pressure. Crude dimethylated product was purified by flash chromatography (DCM/MeOH gradient).

The pKa for this compound as measured by TNS dye assay was 5.3.

### Example 7

### Preparation of C18:2-DAP(NH₃⁺Cl⁻)—C18:1

C18:2-DAP-C18:1 was synthesized following methods as for Example 3 with appropriate components.

### Example 8

### Preparation of C18:3-DAP(NH₃⁺Cl⁻)—C18:3

C18:3-DAP-C18:3 was synthesized following methods as for Example 3 with appropriate components. The pKa for this compound as measured by TNS dye assay was 5.7.

### Example 9

### Preparation of C18:1-DAB-C18:1

C18:1-DAB-C18:1 was synthesized following methods as for Example 3 with appropriate components.

### Example 10

### Preparation of C18:1-DAB(N-Me,N-Me)-C18:1

C18:1-DAB(Me,Me)-C18:1 was synthesized following methods as for Example 3 with appropriate components and the following additional step: To crude hydrochloride of amino acid with 2 aliphatic chains from the previous step dissolved in MeOH, 10 eq of AcOH and 10 eq of 37% formaldehyde were added and reaction solution was heated up to very gentle reflux. Then 10 eq of sodium triacetoxyborohydride were added. The reaction was stirred for 30 min, cooled down to room temperature and worked up by the addition of H₂O and saturated NaHCO3. Product was extracted with DCM and organic layer was dried over MgSO4, filtered and organic solvent was removed under reduced pressure. Crude dimethylated product was purified by flash chromatography (DCM/MeOH gradient).

The pKa's for this compound as measured by TNS dye assay were 3.9 and 7.3.

### Example 11

### Preparation of C18:2-DAB-C18:2 and Thermal Properties

C18:2-DAB-C18:2 was synthesized following methods as for Example 3 with appropriate components.

Differential scanning calorimetry was used to distinguish the properties of the activity-generating delivery molecule of this invention C(18:2)-DAB-C(18:2) from other compounds.

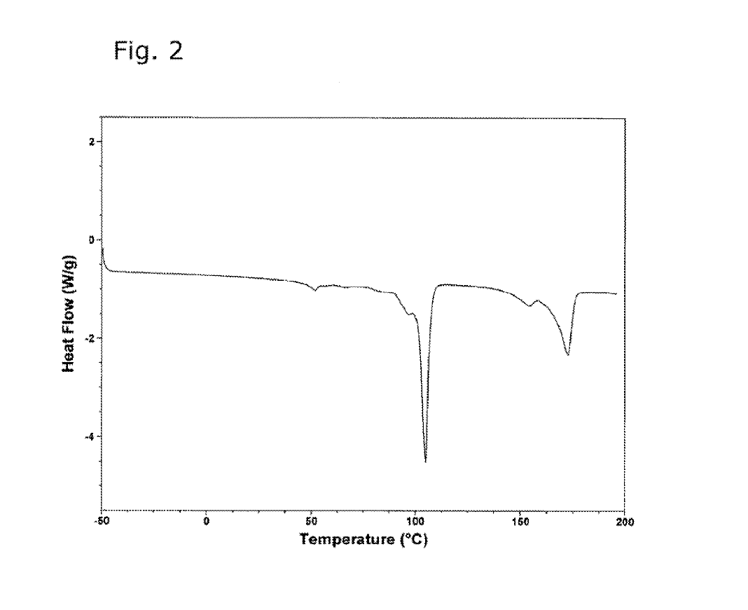

For example, in FIG. 2 is shown a chart of the 2nd melting behavior and thermal phase properties assessed by differential scanning calorimetry of the compound CH₃(CH₂)₁₆(CO)-norArg-NH(CH₂)₁₇CH₃ (See US 2008-0317839 A1). The large peaks in FIG. 2 indicate the presence of significant thermal or melting transitions.

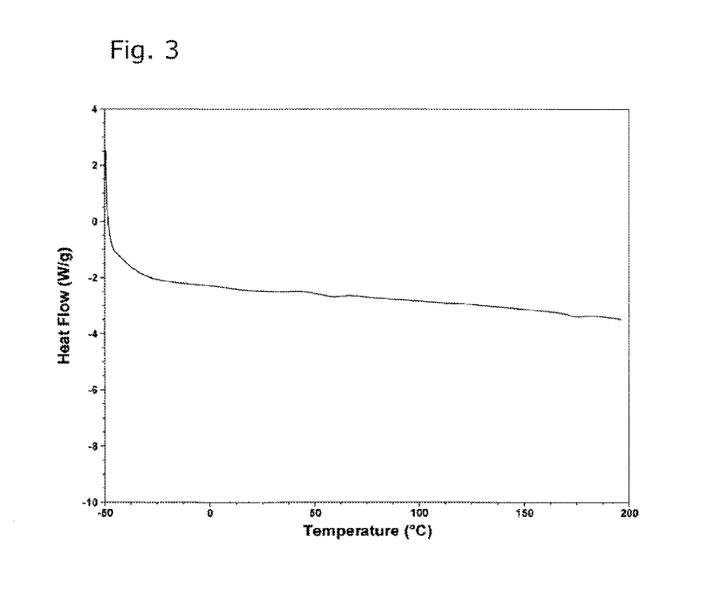

By comparison, in FIG. 3 is shown a chart of the 2nd melting behavior and thermal phase properties of the compound C(18:2)oleoyl-DAB-C(18:2)alkenylamino assessed by differential scanning calorimetry, which represents an embodiment of this invention. The DSC scan in FIG. 3 reveals the complete lack of thermal transition peaks in the compound.

### Example 12

### Preparation of C18:2-DAB(Me,Me)-C18:2

C18:2-DAB(Me,Me)-C18:2 was synthesized following methods as for Example 3 with appropriate components and the following additional step: To crude hydrochloride of amino acid with 2 aliphatic chains from the previous step dissolved in MeOH, 10 eq of AcOH and 10 eq of 37% formaldehyde were added and reaction solution was heated up to very gentle reflux. Then 10 eq of sodium triacetoxyborohydride were added. The reaction was stirred for 30 min, cooled down to room temperature and worked up by the addition of H₂O and saturated NaHCO3. Product was extracted with DCM and organic layer was dried over MgSO4, filtered and organic solvent was removed under reduced pressure. Crude dimethylated product was purified by flash chromatography (DCM/MeOH gradient).

### Example 13

### Preparation of C18:2-Orn-C18:2

C18:2-Orn-C18:2 was synthesized following methods as for Example 3 with appropriate components.

### Example 14

### Preparation of C18:2-Lys(NH₃⁺Cl⁻)—C18:2

C18:2-Lys-C18:2 was synthesized following methods as for Example 3 with appropriate components.

### Example 15

### Preparation of C18:1-norArg-C18:1

C18:1-norArg-C18:1 was synthesized as follows. Fmoc-N γ-Boc-L-2,3-diaminobutyric acid was dissolved in dichloromethane (DCM), 2eq of diisopropylethyl amine (DIPEA) and the resulting solution was added to 2-chlorotrityl choride resin. After one hour, the resin was washed with DCM and Fmoc group was removed by treatment with 20% piperidine in DMF yielding the free α-amine. Oleic acid was preactivated with 2-(6-Chloro-1H-benzotriazole-1-yl)-1,1,3,3-tetramethylaminium hexafluorophosphate (HCTU) and 2 equivalents of DIPEA and added to the resin and the reaction was deemed complete by negative Kaiser test. The lipidated compound was cleaved from the resin by multiple treatments with 1% trifluoroacetic acid (TFA) in dichloromethane followed by evaporation under reduced pressure yielding free carboxylate intermediate. The second alkyl chain was attached by preactivating the free carboxyl group with (1-Ethyl-3-(3-dimethyllaminopropyl)-carbodiimide hydrochloride) (EDC) and N-Hydroxybenzotriazole (HOBt) in a 1:1 mixture of DMF and DCM for 10 minutes followed by addition of oleyl amine in same solvent and subsequent stirring for 30 minutes. Crude compound was purified by flash chromatography (Hexane/AcOEt gradient). The pure dialkylated intermediate was dissolved in 1M HCl/ethyl acetate solution and the Boc group was removed within one hour followed by removal of the solvent under reduced pressure. The resulting white solid was taken up in DCM to which was added TEA facilitate dissolution followed by treatment with 1, 3 Di-Boc-2-(trifluoromethylsulfonyl) guanidine for one hour. Upon completion of the reaction DCM was washed with 2 M sodium bisulfate, saturated sodium bicarbonate and dried over MgSO4 and removed under reduced pressure. The resulting residue was dissolved in absolute ethanol and two Boc groups were removed by adding dissolved compound drop wise to 12N HCl. Final product precipitated during reaction and was crystallized from EtOH.

### Example 16

### Preparation of C18:2-norArg-C18:2

C18:2-norArg-C18:2 was synthesized following methods as for Example 15 with appropriate components.

### Example 17

### Preparation of C18:1-Me-His-C18:1

C18:1-Me-His-C18:1 was synthesized following methods as for Example 3 with appropriate components. The pKa for this compound as measured by TNS dye assay was 3.9.

### Example 18

### Preparation of C18:2-amino-Pro-C18:2

C18:2-amino-Pro-C18:2 was synthesized following methods as for Example 3 with appropriate components.

### Example 19

### Preparation of C18:2-amino-Pro(N,N-diMe)-C18:2

C18:2-amino-Pro(Me,Me)-C18:2 was synthesized following methods as for Example 3 with appropriate components and the following additional step: To crude hydrochloride of amino acid with 2 aliphatic chains from the previous step dissolved in MeOH, 10 eq of AcOH and 10 eq of 37% formaldehyde were added and reaction solution was heated up to very gentle reflux. Then 10 eq of sodium triacetoxyborohydride were added. The reaction was stirred for 30 min, cooled down to room temperature and worked up by the addition of H₂O and saturated NaHCO3. Product was extracted with DCM and organic layer was dried over MgSO4, filtered and organic solvent was removed under reduced pressure. Crude dimethylated product was purified by flash chromatography (DCM/MeOH gradient).

The pKa for this compound as measured by TNS dye assay was 6.6.

### Example 20

### TNS Assay for pKa

To measure pH responsive fluorescence, a TNS/Liposome reaction mixture was prepared as follows: 16 μL of TNS at 1 mg/mL (dissolved in 20% DMF), 160 μL of liposome test sample at 1 mM and 3824 μL of H20. Briefly, in a Costar 96 well plate; 100 μL/well of 2× universal buffer was added (50 mM Citrate; 40 mM sodium phosphate; 40 mM ammonium acetate; 300 mM NaCl, the 2× buffer was titrated to different pHs, at 0.5 pH increments from pH 3.0 to pH 11.0 using NaOH or HCl). To each well containing the 100 μL of universal buffer, 100 μL of the TNS/Liposome reaction mixture is then added to achieve a final volume of 200 μL, a TNS concentration of 5.92 μM and a final lipid concentration of 20 μM per well. After 30 min. at 37° C. the fluorescence was read at an excitation wavelength of 322 nm and emission wavelength of 431 nm. The pKa was determined at the pH corresponding to the midpoint between the maximum and the minimum fluorescence intensity, utilizing sigmoidal fit software.

TNS dye (2,6-TNS (2-(p-toluidinyl)naphthalene-6-sulfonic acid, sodium salt; Invitrogen T53; MW 335.4).

### Example 21

### DSC Measurement for Melting Behavior

The 2nd melting behavior of activity-generating delivery molecules was assessed on a TA Instruments Q200 Differential Scanning Calorimeter in a heat/cool/heat cycle by weighing 0.5 to 1.5 mg of the powder into an aluminum pan and heating to 175° C. at 20° C./min., cooling to −50° C. at 10° C./min., and heating to 200° C. at 20° C./min.

Without intending to be bound by any particular theory, the melting behavior of an activity-generating delivery molecule may relate to its ability to form lamellar, bilayer, or other ordered structures which can be useful for carrying an active agent to interact with, and enter a cellular compartment to generate pharmacological or biological activity. A balance may be desirable between highly ordered structures which exhibit significant thermotropic phases or transitions, and less ordered structures which exhibit little or no thermotropic transitions. A substance having less ordered structures may provide greater membrane fusion ability which can be needed for delivery of the active agent to a cell. Thus, differential scanning calorimetry can be used to distinguish the properties of compounds with certain significant thermotropic phases or transitions from compounds with different properties and less significant thermotropic phases or transitions.

### Example 22

### Methods for Preparing an RNA-Containing Nanoparticle Formulation

This example describes embodiments of methods for making an RNA-containing nanoparticle formulation. Some materials used in the method are summarized below:

C18:1-norArg-C16 (Palmitoyl Oleyl nor-Arginine, PONA) (Marina Biotech, Inc.) (formula weight 683.3)

1,2-Dimyristoyl-sn-Glycero-3-Phosphoethanolamine-N-[Methoxy(Polyethylene glycol)-2000] (Ammonium Salt) (DMPE-PEG2k) (Genzyme Pharmaceuticals, Cambridge, Mass.)

Cholesterol (Solvay Pharmaceuticals)

Cholesteryl-hemisuccinate (CHEMS) GMP (Merck Eprova AG)

Ethanol (absolute, 200 proof); Sterile water for injection

Sodium phosphate: monobasic, anhydrous, dibasic, anhydrous

Sucrose, 99+%

5 N sodium hydroxide; 2 N hydrochloric acid; Glacial acetic acid

Tromethamine (Tris) USP Grade (Research Organics)

150 mL Capacity 0.2 μm filter bottles, PES

Calibrated Rainin 20 μL, 200 μL, and 1 mL pipettors

Iso-disc filter PTFE25-10

Cole-Parmer In-line static mixer

Watson Marlow 520 Di pump; Watson Marlow 523 pump; Filtertec pump

Vivaflow 50 100,00 MWCO PES (Sartorius)

Slide-a-Lyser dialysis cassette 10,000 MWCO (Pierce)

The buffer solution Sucrose Phosphate (SUP) Formulation Buffer (20 mM sodium phosphate, 215 mM sucrose, pH 7.4) was prepared as follows. 2.17 g anhydrous monobasic sodium phosphate and 8.79 g anhydrous dibasic sodium phosphate were added to 3600 mL of Milli-Q DI water in a graduated cylinder and mixed thoroughly with a stir bar. The pH was adjusted with 5N sodium hydroxide or 2N hydrogen chloride to pH 7.4. 294.38 g sucrose was added slowly and dissolved thoroughly. Final water volume was adjusted to 4 L. The solution was filtered with a 0.2 μm filter.

A 25 mM stock solution of nanoparticle-forming molecules in 90% v/v ethanol USP was prepared as follows. 90 mL of ethanol USP (200 proof) was dispensed into a clean autoclaved 100 mL Pyrex bottle. To the ethanol were added successively 1291 umol of C18:1-norArg-C16 (PONA), 721.6 umol of cholesteryl-hemisuccinate (CHEMS) powder, 61.7 umol of DMPE-PEG2K powder, and 515 umol of cholesterol. The ingredients were each added to the solution and mixed thoroughly with a stir bar. The mixture was sonicated for 15 minutes. 10 mL of sterile water for injection USP was added with thorough mixing. The stock solution was filtered through an TSO-DTSC filter PTFE-25 mm, 1 um pore size. The stock solution was stored at 80° C. and analyzed for C18:1-norArg-C16 and lipid components by Reverse Phase HPLC with Evaporative Light Scattering Detection.

An siRNA stock solution was prepared in sterile water for injection as follows. 5 mL of sterile water for injection was dispensed into a sterile 15 mL Falcon tube. 100 mg of siRNA powder was added to the tube and vortexed thoroughly. The solution was filtered through a 0.22 uM Millex GP filter unit using a 10 mL syringe. The siRNA solution was stored at −20° C. and tested by OD (A260 and A280) for purity and concentration with 1:1000 dilution.

A Watson Marlow 520Di peristaltic pump was calibrated to a flow rate of 40 mL/min. The pump was set to 210 rpm and disconnected from the tubing. 40 mL of 90% ethanol was pumped through to rinse the line. Ethanol was pumped into a beaker for 15 sec and weighed to determine the flow rate in mL/min. The pump speed was adjusted to provide a flow rate of 40±0.5 mL/min. Pumps for siRNA and sucrose phosphate solutions were calibrated in a similar manner.

Three solutions were used to prepare an siRNA formulation as follows. (a) The first solution for pumping was an siRNA solution. The first solution was made by diluting the siRNA with SUP buffer in a 50 mL conical tube and vortexing thoroughly. (b) The second solution for pumping was a solution of C18:1-norArg-C16 plus three lipids. A mixed lipid stock in 90% ethanol was prepared containing the following lipids: CHEMS, cholesterol, and DMPE-PEG. To the lipid stock was added C18:1-norArg-C16. To the lipid stock was added an aliquot of Tris in sterile water for injection to make a 1:1 molar Tris:CHEMS concentration in the solution. The second solution for pumping was made with the mixed lipid stock by pipetting with a positive displacement pipette into a 50 mL conical tube, diluting with 90% ethanol, and vortexing thoroughly. (c) A third solution for pumping was an SUP buffer solution.

An siRNA formulation was prepared as follows. The first siRNA solution and the second solution of nanoparticle-forming molecules were simultaneously pumped into an impinging stream. The first 1 mL of the effluent impinging stream was discarded, then the siRNA formulation was collected in a vessel. A Watson Marlow 323 pump was used to pump SUP buffer solution into the vessel to adjust the concentration of ethanol to be about 33%. The siRNA formulation in the vessel was incubated with gentle agitation on magnetic stir plate for 1 hr.

After incubation, the formulation was loaded into a Pierce slide-a-lyzer dialysis cassette with 10,000 MWCO, and dialyzed for 12-18 hrs at 4° C. against 100 volumes of SUP.

This example further describes embodiments of methods for making an RNA-containing nanoparticle formulation by tangential flow and diafiltration. A siRNA formulation was provided as described above, except that the last dialysis step was replaced by a tangential flow filtration (TFF) process.

The siRNA formulation was diluted to 10% (v/v) final ethanol concentration under gentle agitation on magnetic stir plate for 2 min.

A TFF system using a Sartorius Vivaflow 50 100,000 MWCO PES membrane was rinsed with 50 mL of 70% ethanol USP, and then re-circulated with 100 mL of 70% ethanol at a pump flow rate of 60 mL/min. The TFF system was rinsed with 50 mL of sterile water and then re-circulated with 100 mL of sterile water at a pump flow rate of 60 mL/min. The TFF system was rinsed with 50 mL of SUP and then re-circulated with 100 mL of SUP at a pump flow rate of 60 mL/min.

The diluted siRNA formulation was loaded into the TFF vessel and concentrated by 5 times to a final siRNA concentration of 0.5 mg/mL (feed pressure ˜20 psi, retentate pressure <0.2 psi and a permeate flow rate of ˜2 mL/min). A maximum of 1 mg of siRNA formulated in the nanoparticle composition was processed per cm² of membrane.

The concentrated siRNA formulation was filtered by diafiltration against 5 volumes of SUP, in which ethanol was removed, at flow rate 2 mL/min.

The concentrated siRNA formulation was further concentrated to the desired volume, at 1 mg/ml siRNA.

This example further describes embodiments of methods for making an RNA-containing nanoparticle formulation by sterile filtration of the siRNA nanoparticle formulation. A siRNA formulation was provided as described above. 10 mL of the siRNA formulation was drawn up in a 10 mL polypropylene syringe, and air bubbles were removed. The siRNA formulation was filtered through a 0.22 uM Millex GP filter unit. 10 mg of siRNA formulation (1 mg siRNA/mL) was filtered though the Millex GP filter unit with moderate pressure on the syringe. 1 mL aliquots of this drug product were stored in 3 mL type I sterile glass vials at 80° C. prior to use.

### Example 23

### Determining In Vitro Gene Silencing Activity of an Activity-Generating Delivery Molecule

The methodology for determining the in vitro gene silencing activity of an activity-generating delivery molecule was as follows: Hep3B cells were transfected in triplicate, 96-well format with a formulation of the activity-generating delivery molecule and a UsiRNA against ApoB. After 24 h, cellular RNA was prepared and evaluated by quantitative RT-PCR for target ApoB and the normalizer 36B4 or GAPDH expression levels.

### Example 24

### Formulations of C18:1-Me-his-C18:1 with an Active RNA Agent

An example formulation containing an activity-generating delivery molecule of this invention is shown in Table 1.

TABLE 1
Formulations of C18:1-Me-His-C18:1 with an active RNA agent

| | | | | Neutral |
|---|---|---|---|---|
| | Activity-generating | Anionic | Neutral | PEGylated |
| No. | delivery molecule | Lipid | Lipid(s) | Lipid |
| 1 | C18:1-Me-His-C18:1 | — | DSPC | — |
| | 50 mole % | | 50 mole % | |
| 2 | C18:1-Me-His-C18:l | — | CHOL | — |
| | 50 mole % | | 50 mole % | |

### Example 25

### Formulations of C18:1-DAP-C18:1 with an Active RNA Agent

Example formulations containing an activity-generating delivery molecule of this invention are shown in Table 2.

TABLE 2
Formulations of C18:1-DAP-C18:1 molecules
with an active RNA agent

| | | | | Neutral |
|---|---|---|---|---|
| | Activity-generating | Anionic | Neutral | PEGylated |
| No. | delivery molecule | Lipid | Lipid(s) | Lipid |
| 1 | C18:1-DAP-C18:1 | CHEMS | — | DMPE-PEG2K |
| | 65.3 mole % | 32.7 mole % | | 2 mole % |
| 2 | C18:1-DAP-C18:1 | CHEMS | — | DMPE-PEG2K |
| | | 32.7 mole % | | 2 mole % |
| 3 | C18:1-DAP-C18:1 | CHEMS | CHOL | DMPE-PEG2K |
| | | 30 mole % | 23 mole % | 2 mole % |
| 4 | C18:1-DAP-C18:1 | CHEMS | CHOL | DMPE-PEG2K |
| | | 30 mole % | 23 mole % | 2 mole % |

### Example 26

### In Vitro Activity Generated with Formulations of C18:2-aminoPro(N-Me,N-Me)-C18:2 with a UsiRNA

Example formulations containing an activity-generating delivery molecule of this invention are shown in Table 3. The in vitro activity was generated with a UsiRNA against ApoB in Hep3B cells.

TABLE 3
Gene knockdown activity generated with C18:2-aminoPro(N—Me,N—Me)-
C18:2 molecules with a UsiRNA

| Activity-generating | | | | | In vitro Gene |
|---|---|---|---|---|---|
| delivery molecule | Mole % | lipid | Mole % | UsiRNA | Knockdown % |
| C18:2-aminoPro(N—Me,N—Me)-C18:2 | 75.00 | CHOL | 25.00 | DX10008 | 59 |
| C18:2-aminoPro(N—Me,N—Me)-C18:2 | 66.66 | CHOL | 33.33 | DX10008 | 53 |
| C18:2-aminoPro(N—Me,N—Me)-C18:2 | 50.00 | CHOL | 50.00 | DX10008 | 62 |
| C18:2-aminoPro(N—Me,N—Me)-C18:2 | 33.33 | CHOL | 66.66 | DX10008 | 72 |
| C18:2-aminoPro(N—Me,N—Me)-C18:2 | 25.00 | CHOL | 75.00 | DX10008 | 79 |
| C18:2-aminoPro(N—Me,N—Me)-C18:2 | 75.00 | DOPE | 25.00 | DX10008 | 68 |
| C18:2-aminoPro(N—Me,N—Me)-C18:2 | 66.66 | DOPE | 33.33 | DX10008 | 73 |
| C18:2-aminoPro(N—Me,N—Me)-C18:2 | 50.00 | DOPE | 50.00 | DX10008 | 82 |
| C18:2-aminoPro(N—Me,N—Me)-C18:2 | 33.33 | DOPE | 66.66 | DX10008 | 74 |
| C18:2-aminoPro(N—Me,N—Me)-C18:2 | 25.00 | DOPE | 75.00 | DX10008 | 66 |

### Example 27

### In Vitro Activity Generated with Formulations of C18:2-Dap(N-Me,N-Me)-C18:1 with a UsiRNA

Example formulations containing an activity-generating delivery molecule of this invention are shown in Table 4. The in vitro activity was generated with a UsiRNA against ApoB in Hep3B cells.

TABLE 4
Gene knockdown activity generated with C18:2-Dap(N—Me,N—Me)-
C18:1 molecules with a UsiRNA agent

| Activity-generating | | | | | In vitro Gene |
|---|---|---|---|---|---|
| delivery molecule | Mole % | lipid | Mole % | UsiRNA | Knockdown % |
| C18:2-Dap(N—Me,N—Me)-C18:1 | 66.66 | CHOL | 33.33 | DX10008 | 91 |
| C18:2-Dap(N—Me,N—Me)-C18:1 | 50.00 | CHOL | 50.00 | DX10008 | 88 |
| C18:2-Dap(N—Me,N—Me)-C18:1 | 33.33 | CHOL | 66.66 | DX10008 | 91 |
| C18:2-Dap(N—Me,N—Me)-C18:1 | 25.00 | CHOL | 75.00 | DX10008 | 90 |
| C18:2-Dap(N—Me,N—Me)-C18:1 | 75.00 | DOPE | 25.00 | DX10008 | 84 |
| C18:2-Dap(N—Me,N—Me)-C18:1 | 66.66 | DOPE | 33.33 | DX10008 | 85 |
| C18:2-Dap(N—Me,N—Me)-C18:1 | 50.00 | DOPE | 50.00 | DX10008 | 84 |
| C18:2-Dap(N—Me,N—Me)-C18:1 | 33.33 | DOPE | 66.66 | DX10008 | 87 |
| C18:2-Dap(N—Me,N—Me)-C18:1 | 25.00 | DOPE | 75.00 | DX10008 | 86 |

### Example 28

### In Vitro Activity Generated with Formulations of C18:2-DAP (N-Me,N-Me)-C18:2 with a UsiRNA

Example formulations containing an activity-generating delivery molecule of this invention are shown in Table 5. The in vitro activity was generated with a UsiRNA against ApoB in Hep3B cells.

TABLE 5
Gene knockdown activity generated with C18:2-DAP (N—Me,N—Me)-
C18:2 molecules with a UsiRNA agent

| Activity-generating | | | | | In vitro Gene |
|---|---|---|---|---|---|
| delivery molecule | Mole % | lipid | Mole % | UsiRNA | Knockdown % |
| C18:2-DAP (N—Me,N—Me)-C18:2 | 75.00 | CHOL | 25.00 | DX10008 | — |
| C18:2-DAP (N—Me,N—Me)-C18:2 | 66.66 | CHOL | 33.33 | DX10008 | 19 |
| C18:2-DAP (N—Me,N—Me)-C18:2 | 50.00 | CHOL | 50.00 | DX10008 | 19 |
| C18:2-DAP (N—Me,N—Me)-C18:2 | 33.33 | CHOL | 66.66 | DX10008 | — |
| C18:2-DAP (N—Me,N—Me)-C18:2 | 25.00 | CHOL | 75.00 | DX10008 | 6 |
| C18:2-DAP (N—Me,N—Me)-C18:2 | 75.00 | DOPE | 25.00 | DX10008 | 18 |
| C18:2-DAP (N—Me,N—Me)-C18:2 | 66.66 | DOPE | 33.33 | DX10008 | 81 |
| C18:2-DAP (N—Me,N—Me)-C18:2 | 50.00 | DOPE | 50.00 | DX10008 | 87 |
| C18:2-DAP (N—Me,N—Me)-C18:2 | 33.33 | DOPE | 66.66 | DX10008 | 86 |

### Example 29

### In Vitro Activity Generated with Formulations of C18:1-DAP(NH3+Cl−)—C18:1 with a UsiRNA

Example formulations containing an activity-generating delivery molecule of this invention are shown in Table 6. The in vitro activity was generated with a UsiRNA against ApoB in Hep3B cells.

TABLE 6
Gene knockdown activity generated with C18:1-DAP(NH3 +
Cl—)-C18:l molecules with a UsiRNA agent

| Activity-generating | | | | | In vitro Gene |
|---|---|---|---|---|---|
| delivery molecule | Mole % | lipid | Mole % | UsiRNA | Knockdown % |
| C18:1-DAP(NH3 + Cl—)-C18:1 | 75.00 | CHOL | 25.00 | DX10008 | 79 |
| C18:1-DAP(NH3 + Cl—)-C18:1 | 66.66 | CHOL | 33.33 | DX10008 | 76 |
| C18:1-DAP(NH3 + Cl—)-C18:1 | 50.00 | CHOL | 50.00 | DX10008 | 73 |
| C18:1-DAP(NH3 + Cl—)-C18:1 | 33.33 | CHOL | 66.66 | DX10008 | 80 |
| C18:1-DAP(NH3 + Cl—)-C18:1 | 25.00 | CHOL | 75.00 | DX10008 | 83 |
| C18:1-DAP(NH3 + Cl—)-C18:1 | 75.00 | DOPE | 25.00 | DX10008 | 77 |
| C18:1-DAP(NH3 + Cl—)-C18:1 | 66.66 | DOPE | 33.33 | DX10008 | 72 |
| C18:1-DAP(NH3 + Cl—)-C18:1 | 50.00 | DOPE | 50.00 | DX10008 | 82 |
| C18:1-DAP(NH3 + Cl—)-C18:1 | 33.33 | DOPE | 66.66 | DX10008 | 83 |
| C18:1-DAP(NH3 + Cl—)-C18:1 | 25.00 | DOPE | 75.00 | DX10008 | 81 |

### Example 30

### In Vitro Activity Generated with Formulations of C18:2-Dap(NH3+Cl−)—C18:1 with a UsiRNA

Example formulations containing an activity-generating delivery molecule of this invention are shown in Table 7. The in vitro activity was generated with a UsiRNA against ApoB in Hep3B cells.

TABLE 7
Gene knockdown activity generated with C18:2-Dap(NH3 +
Cl—)-C18:1 with a UsiRNA

| Activity-generating | | | | | In vitro Gene |
|---|---|---|---|---|---|
| delivery molecule | Mole % | lipid | Mole % | UsiRNA | Knockdown % |
| C18:2-Dap(NH3 + Cl—)-C18:1 | 75.00 | CHOL | 25.00 | DX10008 | 52 |
| C18:2-Dap(NH3 + Cl—)-C18:1 | 66.66 | CHOL | 33.33 | DX10008 | 61 |
| C18:2-Dap(NH3 + Cl—)-C18:1 | 50.00 | CHOL | 50.00 | DX10008 | 56 |
| C18:2-Dap(NH3 + Cl—)-C18:1 | 33.33 | CHOL | 66.66 | DX10008 | 62 |
| C18:2-Dap(NH3 + Cl—)-C18:1 | 25.00 | CHOL | 75.00 | DX10008 | 64 |
| C18:2-Dap(NH3 + Cl—)-C18:1 | 75.00 | DOPE | 25.00 | DX10008 | 48 |
| C18:2-Dap(NH3 + Cl—)-C18:1 | 66.66 | DOPE | 33.33 | DX10008 | 69 |
| C18:2-Dap(NH3 + Cl—)-C18:1 | 50.00 | DOPE | 50.00 | DX10008 | 83 |
| C18:2-Dap(NH3 + Cl—)-C18:1 | 33.33 | DOPE | 66.66 | DX10008 | 83 |
| C18:2-Dap(NH3 + Cl—)-C18:1 | 25.00 | DOPE | 75.00 | DX10008 | 85 |

### Example 31

### In Vitro Activity Generated with Formulations of C18:2-DAP(NH₃⁺Cl⁻)—C18:1 with a UsiRNA

Example formulations containing an activity-generating delivery molecule of this invention are shown in Table 8. The in vitro activity was generated with a UsiRNA against ApoB in Hep3B cells.

TABLE 8
Gene knockdown activity generated with C18:2-aminoPro(N—Me,N—Me)-
C18:2 with a UsiRNA

| Activity-generating | | | | | In vitro Gene |
|---|---|---|---|---|---|
| delivery molecule | Mole % | lipid | Mole % | UsiRNA | Knockdown % |
| C18:2-aminoPro(N—Me,N—Me)-C18:2 | 75.00 | CHOL | 25.00 | DX10008 | 82 |
| C18:2-aminoPro(N—Me,N—Me)-C18:2 | 66.66 | CHOL | 33.33 | DX10008 | 75 |
| C18:2-aminoPro(N—Me,N—Me)-C18:2 | 50.00 | CHOL | 50.00 | DX10008 | 73 |
| C18:2-aminoPro(N—Me,N—Me)-C18:2 | 33.33 | CHOL | 66.66 | DX10008 | 67 |
| C18:2-aminoPro(N—Me,N—Me)-C18:2 | 25.00 | CHOL | 75.00 | DX10008 | 58 |
| C18:2-aminoPro(N—Me,N—Me)-C18:2 | 75.00 | DOPE | 25.00 | DX10008 | 42 |
| C18:2-aminoPro(N—Me,N—Me)-C18:2 | 66.66 | DOPE | 33.33 | DX10008 | 43 |
| C18:2-aminoPro(N—Me,N—Me)-C18:2 | 50.00 | DOPE | 50.00 | DX10008 | 48 |
| C18:2-aminoPro(N—Me,N—Me)-C18:2 | 33.33 | DOPE | 66.66 | DX10008 | 70 |
| C18:2-aminoPro(N—Me,N—Me)-C18:2 | 25.00 | DOPE | 75.00 | DX10008 | 78 |

### Example 32

### In Vivo Activity Generated with Formulations of C18:2-aminoPro(N,N-diMe)-C18:2 with a UsiRNA

Example formulations containing an activity-generating delivery molecule of this invention are shown in Table 9. In vivo gene knockdown activity was generated in Balb/c mice administered by tail-vein injection with a formulation including the activity-generating delivery molecule and a UsiRNA against Factor VII mRNA.

TABLE 9
Gene knockdown activity generated with C18:2-
aminoPro(N,N-diMe)-C18:2 with a UsiRNA

| Activity-generating | | | In vivo Gene |
|---|---|---|---|
| delivery molecule | lipid | PEG-lipid | Knockdown % |
| C18:2-aminoPro(N,N-diMe)-C18:2 | CHOL | DMPE-PEG2k | 68 |
| 49 mole % | 49 mole % | 2 mole % | |
| C18:2-aminoPro(N,N-diMe)-C18:2 | CHOL | DMPE-PEG2k | 48 |
| 65.3 mole % | 32.7 mole % | 2 mole % | |

1. A compound comprising an amino acid having a long chain alkenoyl group at the N-terminus and a long chain alkenylamino group at the C-terminus, wherein each long chain group has from 12 to 24 carbon atoms and one or more carbon-carbon double bonds.

2. The compound of claim 1, wherein at least one long chain group has two or more carbon-carbon double bonds.

3. The compound of claim 1, comprising the structure shown in Formula I:
R³—(C═O)-Xaa-NH—R⁴ Formula I
wherein
Xaa is any D- or L-amino acid residue having the general formula —NR^{N}—CR¹R²—(C═O)—, wherein
R¹ is a non-hydrogen, substituted or unsubstituted side chain of an amino acid;
R², R^{N} are independently hydrogen, or an organic group consisting of carbon, oxygen, nitrogen, sulfur, and hydrogen atoms, and having from 1 to 20 carbon atoms, or C(1-5)alkyl, cycloalkyl, cycloalkylalkyl, C(3-5)alkenyl, C(3-5)alkynyl, C(1-5)alkanoyl, C(1-5)alkanoyloxy, C(1-5)alkoxy, C(1-5)alkoxy-C(1-5)alkyl, C(1-5)alkoxy-C(1-5)alkoxy, C(1-5)alkyl-amino-C(1-5)alkyl-, C(1-5)dialkyl-amino-C(1-5)alkyl-, nitro-C(1-5)alkyl, cyano-C(1-5)alkyl, aryl-C(1-5)alkyl, 4-biphenyl-C(1-5)alkyl, carboxyl, or hydroxyl;
R³—(C═O)— is independently a long chain group which may be derived from a naturally-occurring phospholipid, glycolipid, triacylglycerol, glycerophospholipid, sphingolipid, ceramide, sphingomyelin, cerebroside, or ganglioside, wherein the long chain group contains one or more carbon-carbon double bonds; or a substituted or unsubstituted C(12-24)alkenoyl;
—NH—R⁴ is independently a long chain group which may be derived from a naturally-occurring phospholipid, glycolipid, triacylglycerol, glycerophospholipid, sphingolipid, ceramide, sphingomyelin, cerebroside, or ganglioside, wherein the long chain group contains one or more carbon-carbon double bonds; or a substituted or unsubstituted C(12-24)alkenylamino;
and salts thereof.

4. The compound of claim 3, wherein R³—(C═O)— is independently a substituted or unsubstituted C(12-24)alkenoyl and —NH—R⁴ is independently a substituted or unsubstituted C(12-24)alkenylamino.

5. The compound of claim 3, wherein R³,R⁴ are each independently C12alkenyl, C13 alkenyl, C14alkenyl, C15alkenyl, C16alkenyl, C17alkenyl, C18alkenyl, C19alkenyl, C20alkenyl, C21alkenyl, C22alkenyl, C23alkenyl, or C24alkenyl.

6. The compound of claim 3, wherein:
R³—(C═O)— is independently C12alkenoyl, C13alkenoyl, C14alkenoyl, C15alkenoyl, C16alkenoyl, C17alkenoyl, C18alkenoyl, C19alkenoyl, C20alkenoyl, C21alkenoyl, C22alkenoyl, C23alkenoyl, or C24alkenoyl; and
—NH—R⁴ is independently C12alkenylamino, C13alkenylamino, C14alkenylamino, C15alkenylamino, C16alkenylamino, C17alkenylamino, C18alkenylamino, C19alkenylamino, C20alkenylamino, C21alkenylamino, C22alkenylamino, C23alkenylamino, or C24alkenylamino.

7. The compound of claim 3, wherein:
R³—(C═O)— is independently C(12:1)alkenoyl, C(12:2)alkenoyl, C(12:3)alkenoyl, C(14:1)alkenoyl, C(14:2)alkenoyl, C(14:3)alkenoyl, C(16:1)alkenoyl, C(16:2)alkenoyl, C(16:3)alkenoyl, C(18:1)alkenoyl, C(18:2)alkenoyl, C(18:3)alkenoyl, C(18:4)alkenoyl, C(20:1)alkenoyl, C(20:2)alkenoyl, C(20:3)alkenoyl, C(20:4)alkenoyl, C(20:5)alkenoyl, C(22:1)alkenoyl, C(22:4)alkenoyl, or C(22:6)alkenoyl; and
—NH—R⁴ is independently C(12:1)alkenylamino, C(12:2)alkenylamino, C(12:3)alkenylamino, C(14:1)alkenylamino, C(14:2)alkenylamino, C(14:3)alkenylamino, C(16:1)alkenylamino, C(16:2)alkenylamino, C(16:3)alkenylamino, C(18:1)alkenylamino, C(18:2)alkenylamino, C(18:3)alkenylamino, C(18:4)alkenylamino, C(20:1)alkenylamino, C(20:2)alkenylamino, C(20:3)alkenylamino, C(20:4)alkenylamino, C(20:5)alkenylamino, C(22:1)alkenylamino, C(22:4)alkenylamino, or C(22:6)alkenylamino.

8. The compound of claim 3, wherein:
R³—(C═O)— is independently C(14:1(5))alkenoyl, C(14:1(9))alkenoyl, C(16:1(7))alkenoyl, C(16:1(9))alkenoyl, C(18:1(3))alkenoyl, C(18:1(5))alkenoyl, C(18:1(7))alkenoyl, C(18:1(9))alkenoyl, C(18:1(11))alkenoyl, C(18:1(12))alkenoyl, C(18:2(9,12))alkenoyl, C(18:2(9,11))alkenoyl, C(18:3(9,12,15))alkenoyl, C(18:3(6,9,12))alkenoyl, C(18:3(9,11,13))alkenoyl, C(18:4(6,9,12,15))alkenoyl, C(18:4(9,11,13,15))alkenoyl, C(20:1(9))alkenoyl, C(20:1(11))alkenoyl, C(20:2(8,11))alkenoyl, C(20:2(5,8))alkenoyl, C(20:2(11,14))alkenoyl, C(20:3(5,8,11))alkenoyl, C(20:4(5,8,11,14))alkenoyl, C(20:4(7,10,13,16))alkenoyl, C(20:5(5,8,11,14,17))alkenoyl, C(20:6(4,7,10,13,16,19))alkenoyl, C(22:1(9))alkenoyl, C(22:1(13))alkenoyl, or C(24:1(9))alkenoyl; and
—NH—R⁴ is independently C(14:1(5))alkenylamino, C(14:1(9))alkenylamino, C(16:1(7))alkenylamino, C(16:1(9))alkenylamino, C(18:1(3))alkenylamino, C(18:1(5))alkenylamino, C(18:1(7))alkenylamino, C(18:1(9))alkenylamino, C(18:1(11))alkenylamino, C(18:1(12))alkenylamino, C(18:2(9,12))alkenylamino, C(18:2(9,11))alkenylamino, C(18:3(9,12,15))alkenylamino, C(18:3(6,9,12))alkenylamino, C(18:3(9,11,13))alkenylamino, C(18:4(6,9,12,15))alkenylamino, C(18:4(9,11,13,15))alkenylamino, C(20:1(9))alkenylamino, C(20:1(11))alkenylamino, C(20:2(8,11))alkenylamino, C(20:2(5,8))alkenylamino, C(20:2(11,14))alkenylamino, C(20:3(5,8,11))alkenylamino, C(20:4(5,8,11,14))alkenylamino, C(20:4(7,10,13,16))alkenylamino, C(20:5(5,8,11,14,17))alkenylamino, C(20:6(4,7,10,13,16,19))alkenylamino, C(22:1(9))alkenylamino, C(22:1(13))alkenylamino, or C(24:1(9))alkenylamino.

9. The compound of claim 3, selected from (18:1(3))-DAA-(18:1(3)), (18:1(5))-DAA-(18:1(5)), (18:1(7))-DAA-(18:1(7)), (18:1(9))-DAA-(18:1(9)), (18:1(11))-DAA-(18:1(11)), (18:1(12))-DAA-(18:1(12)), (18:1(3))-DAA-(18:1(5)), (18:1(3))-DAA-(18:1(7)), (18:1(3))-DAA-(18:1(9)), (18:1(3))-DAA-(18:1(11)), (18:1(3))-DAA-(18:1(12)), (18:1(5))-DAA-(18:1(7)), (18:1(5))-DAA-(18:1(9)), (18:1(5))-DAA-(18:1(11)), (18:1(5))-DAA-(18:1(12)), (18:1(7))-DAA-(18:1(9)), (18:1(7))-DAA-(18:1(11)), (18:1(7))-DAA-(18:1(12)), (18:1(9))-DAA-(18:1(11)), (18:1(9))-DAA-(18:1(12)), (18:1(11))-DAA-(18:1(12)), (18:1(3))-DAA-(18:2(9,12)), (18:1(5))-DAA-(18:2(9,12)), (18:1(7))-DAA-(18:2(9,12)), (18:1(9))-DAA-(18:2(9,12)), (18:1(11))-DAA-(18:2(9,12)), (18:1(12))-DAA-(18:2(9,12)), (18:2(9,12))-DAA-(18:1(3)), (18:2(9,12))-DAA-(18:1(5)), (18:2(9,12))-DAA-(18:1(7)), (18:2(9,12))-DAA-(18:1(9)), (18:2(9,12))-DAA-(18:1(11)), (18:2(9,12))-DAA-(18:1(12)), (18:2(9,12))-DAA-(18:2(9,12)), and a cationic form of any of the foregoing.

10. The compound of claim 3, selected from (18:1(3))-DAP-(18:1(3)), (18:1(5))-DAP-(18:1(5)), (18:1(7))-DAP-(18:1(7)), (18:1(9))-DAP-(18:1(9)), (18:1(11))-DAP-(18:1(11)), (18:1(12))-DAP-(18:1(12)), (18:1(3))-DAP-(18:1(5)), (18:1(3))-DAP-(18:1(7)), (18:1(3))-DAP-(18:1(9)), (18:1(3))-DAP-(18:1(11)), (18:1(3))-DAP-(18:1(12)), (18:1(5))-DAP-(18:1(7)), (18:1(5))-DAP-(18:1(9)), (18:1(5))-DAP-(18:1(11)), (18:1(5))-DAP-(18:1(12)), (18:1(7))-DAP-(18:1(9)), (18:1(7))-DAP-(18:1(11)), (18:1(7))-DAP-(18:1(12)), (18:1(9))-DAP-(18:1(11)), (18:1(9))-DAP-(18:1(12)), (18:1(11))-DAP-(18:1(12)), (18:1(3))-DAP-(18:2(9,12)), (18:1(5))-DAP-(18:2(9,12)), (18:1(7))-DAP-(18:2(9,12)), (18:1(9))-DAP-(18:2(9,12)), (18:1(11))-DAP-(18:2(9,12)), (18:1(12))-DAP-(18:2(9,12)), (18:2(9,12))-DAP-(18:1(3)), (18:2(9,12))-DAP-(18:1(5)), (18:2(9,12))-DAP-(18:1(7)), (18:2(9,12))-DAP-(18:1(9)), (18:2(9,12))-DAP-(18:1(11)), (18:2(9,12))-DAP-(18:1(12)), (18:2(9,12))-DAP-(18:2(9,12)), and a cationic form of any of the foregoing.

11. The compound of claim 3, selected from (18:1(3))-DAB-(18:1(3)), (18:1(5))-DAB-(18:1(5)), (18:1(7))-DAB-(18:1(7)), (18:1(9))-DAB-(18:1(9)), (18:1(11))-DAB-(18:1(11)), (18:1(12))-DAB-(18:1(12)), (18:1(3))-DAB-(18:1(5)), (18:1(3))-DAB-(18:1(7)), (18:1(3))-DAB-(18:1(9)), (18:1(3))-DAB-(18:1(11)), (18:1(3))-DAB-(18:1(12)), (18:1(5))-DAB-(18:1(7)), (18:1(5))-DAB-(18:1(9)), (18:1(5))-DAB-(18:1(11)), (18:1(5))-DAB-(18:1(12)), (18:1(7))-DAB-(18:1(9)), (18:1(7))-DAB-(18:1(11)), (18:1(7))-DAB-(18:1(12)), (18:1(9))-DAB-(18:1(11)), (18:1(9))-DAB-(18:1(12)), (18:1(11))-DAB-(18:1(12)), (18:1(3))-DAB-(18:2(9,12)), (18:1(5))-DAB-(18:2(9,12)), (18:1(7))-DAB-(18:2(9,12)), (18:1(9))-DAB-(18:2(9,12)), (18:1(11))-DAB-(18:2(9,12)), (18:1(12))-DAB-(18:2(9,12)), (18:2(9,12))-DAB-(18:1(3)), (18:2(9,12))-DAB-(18:1(5)), (18:2(9,12))-DAB-(18:1(7)), (18:2(9,12))-DAB-(18:1(9)), (18:2(9,12))-DAB-(18:1(11)), (18:2(9,12))-DAB-(18:1(12)), (18:2(9,12))-DAB-(18:2(9,12)), and a cationic form of any of the foregoing.

12. The compound of claim 3, selected from (18:1(3))-Orn-(18:1(3)), (18:1(5))-Orn-(18:1(5)), (18:1(7))-Orn-(18:1(7)), (18:1(9))-Orn-(18:1(9)), (18:1(11))-Orn-(18:1(11)), (18:1(12))-Orn-(18:1(12)), (18:1(3))-Orn-(18:1(5)), (18:1(3))-Orn-(18:1(7)), (18:1(3))-Orn-(18:1(9)), (18:1(3))-Orn-(18:1(11)), (18:1(3))-Orn-(18:1(12)), (18:1(5))-Orn-(18:1(7)), (18:1(5))-Orn-(18:1(9)), (18:1(5))-Orn-(18:1(11)), (18:1(5))-Orn-(18:1(12)), (18:1(7))-Orn-(18:1(9)), (18:1(7))-Orn-(18:1(11)), (18:1(7))-Orn-(18:1(12)), (18:1(9))-Orn-(18:1(11)), (18:1(9))-Orn-(18:1(12)), (18:1(11))-Orn-(18:1(12)), (18:1(3))-Orn-(18:2(9,12)), (18:1(5))-Orn-(18:2(9,12)), (18:1(7))-Orn-(18:2(9,12)), (18:1(9))-Orn-(18:2(9,12)), (18:1(11))-Orn-(18:2(9,12)), (18:1(12))-Orn-(18:2(9,12)), (18:2(9,12))-Orn-(18:1(3)), (18:2(9,12))-Orn-(18:1(5)), (18:2(9,12))-Orn-(18:1(7)), (18:2(9,12))-Orn-(18:1(9)), (18:2(9,12))-Orn-(18:1(11)), (18:2(9,12))-Orn-(18:1(12)), (18:2(9,12))-Orn-(18:2(9,12)), and a cationic form of any of the foregoing.

13. The compound of claim 3, selected from (18:1(3))-Lys-(18:1(3)), (18:1(5))-Lys-(18:1(5)), (18:1(7))-Lys-(18:1(7)), (18:1(9))-Lys-(18:1(9)), (18:1(11))-Lys-(18:1(11)), (18:1(12))-Lys-(18:1(12)), (18:1(3))-Lys-(18:1(5)), (18:1(3))-Lys-(18:1(7)), (18:1(3))-Lys-(18:1(9)), (18:1(3))-Lys-(18:1(11)), (18:1(3))-Lys-(18:1(12)), (18:1(5))-Lys-(18:1(7)), (18:1(5))-Lys-(18:1(9)), (18:1(5))-Lys-(18:1(11)), (18:1(5))-Lys-(18:1(12)), (18:1(7))-Lys-(18:1(9)), (18:1(7))-Lys-(18:1(11)), (18:1(7))-Lys-(18:1(12)), (18:1(9))-Lys-(18:1(11)), (18:1(9))-Lys-(18:1(12)), (18:1(11))-Lys-(18:1(12)), (18:1(3))-Lys-(18:2(9,12)), (18:1(5))-Lys-(18:2(9,12)), (18:1(7))-Lys-(18:2(9,12)), (18:1(9))-Lys-(18:2(9,12)), (18:1(11))-Lys-(18:2(9,12)), (18:1(12))-Lys-(18:2(9,12)), (18:2(9,12))-Lys-(18:1(3)), (18:2(9,12))-Lys-(18:1(5)), (18:2(9,12))-Lys-(18:1(7)), (18:2(9,12))-Lys-(18:1(9)), (18:2(9,12))-Lys-(18:1(11)), (18:2(9,12))-Lys-(18:1(12)), (18:2(9,12))-Lys-(18:2(9,12)), and a cationic form of any of the foregoing.

14. The compound of claim 3, selected from (18:1(3))-norArg-(18:1(3)), (18:1(5))-norArg-(18:1(5)), (18:1(7))-norArg-(18:1(7)), (18:1(9))-norArg-(18:1(9)), (18:1(11))-norArg-(18:1(11)), (18:1(12))-norArg-(18:1(12)), (18:1(3))-norArg-(18:1(5)), (18:1(3))-norArg-(18:1(7)), (18:1(3))-norArg-(18:1(9)), (18:1(3))-norArg-(18:1(11)), (18:1(3))-norArg-(18:1(12)), (18:1(5))-norArg-(18:1(7)), (18:1(5))-norArg-(18:1(9)), (18:1(5))-norArg-(18:1(11)), (18:1(5))-norArg-(18:1(12)), (18:1(7))-norArg-(18:1(9)), (18:1(7))-norArg-(18:1(11)), (18:1(7))-norArg-(18:1(12)), (18:1(9))-norArg-(18:1(11)), (18:1(9))-norArg-(18:1(12)), (18:1(11))-norArg-(18:1(12)), (18:1(3))-norArg-(18:2(9,12)), (18:1(5))-norArg-(18:2(9,12)), (18:1(7))-norArg-(18:2(9,12)), (18:1(9))-norArg-(18:2(9,12)), (18:1(11))-norArg-(18:2(9,12)), (18:1(12))-norArg-(18:2(9,12)), (18:2(9,12))-norArg-(18:1(3)), (18:2(9,12))-norArg-(18:1(5)), (18:2(9,12))-norArg-(18:1(7)), (18:2(9,12))-norArg-(18:1(9)), (18:2(9,12))-norArg-(18:1(11)), (18:2(9,12))-norArg-(18:1(12)), (18:2(9,12))-norArg-(18:2(9,12)), and a cationic form of any of the foregoing.

15. The compound of claim 3, selected from (18:1(3))-His-(18:1(3)), (18:1(5))-His-(18:1(5)), (18:1(7))-His-(18:1(7)), (18:1(9))-His-(18:1(9)), (18:1(11))-His-(18:1(11)), (18:1(12))-His-(18:1(12)), (18:1(3))-His-(18:1(5)), (18:1(3))-His-(18:1(7)), (18:1(3))-His-(18:1(9)), (18:1(3))-His-(18:1(11)), (18:1(3))-His-(18:1(12)), (18:1(5))-His-(18:1(7)), (18:1(5))-His-(18:1(9)), (18:1(5))-His-(18:1(11)), (18:1(5))-His-(18:1(12)), (18:1(7))-His-(18:1(9)), (18:1(7))-His-(18:1(11)), (18:1(7))-His-(18:1(12)), (18:1(9))-His-(18:1(11)), (18:1(9))-His-(18:1(12)), (18:1(11))-His-(18:1(12)), (18:1(3))-His-(18:2(9,12)), (18:1(5))-His-(18:2(9,12)), (18:1(7))-His-(18:2(9,12)), (18:1(9))-His-(18:2(9,12)), (18:1(11))-His-(18:2(9,12)), (18:1(12))-His-(18:2(9,12)), (18:2(9,12))-His-(18:1(3)), (18:2(9,12))-His-(18:1(5)), (18:2(9,12))-His-(18:1(7)), (18:2(9,12))-His-(18:1(9)), (18:2(9,12))-His-(18:1(11)), (18:2(9,12))-His-(18:1(12)), (18:2(9,12))-His-(18:2(9,12)), and a cationic form of any of the foregoing.

16. The compound of claim 3, selected from (18:1(3))-Pro-(18:1(3)), (18:1(5))-Pro-(18:1(5)), (18:1(7))-Pro-(18:1(7)), (18:1(9))-Pro-(18:1(9)), (18:1(11))-Pro-(18:1(11)), (18:1(12))-Pro-(18:1(12)), (18:1(3))-Pro-(18:1(5)), (18:1(3))-Pro-(18:1(7)), (18:1(3))-Pro-(18:1(9)), (18:1(3))-Pro-(18:1(11)), (18:1(3))-Pro-(18:1(12)), (18:1(5))-Pro-(18:1(7)), (18:1(5))-Pro-(18:1(9)), (18:1(5))-Pro-(18:1(11)), (18:1(5))-Pro-(18:1(12)), (18:1(7))-Pro-(18:1(9)), (18:1(7))-Pro-(18:1(11)), (18:1(7))-Pro-(18:1(12)), (18:1(9))-Pro-(18:1(11)), (18:1(9))-Pro-(18:1(12)), (18:1(11))-Pro-(18:1(12)), (18:1(3))-Pro-(18:2(9,12)), (18:1(5))-Pro-(18:2(9,12)), (18:1(7))-Pro-(18:2(9,12)), (18:1(9))-Pro-(18:2(9,12)), (18:1(11))-Pro-(18:2(9,12)), (18:1(12))-Pro-(18:2(9,12)), (18:2(9,12))-Pro-(18:1(3)), (18:2(9,12))-Pro-(18:1(5)), (18:2(9,12))-Pro-(18:1(7)), (18:2(9,12))-Pro-(18:1(9)), (18:2(9,12))-Pro-(18:1(11)), (18:2(9,12))-Pro-(18:1(12)), (18:2(9,12))-Pro-(18:2(9,12)), (18:1(3))-Pro(4-amino)-(18:1(3)), (18:1(5))-Pro(4-amino)-(18:1(5)), (18:1(7))-Pro(4-amino)-(18:1(7)), (18:1(9))-Pro(4-amino)-(18:1(9)), (18:1(11))-Pro(4-amino)-(18:1(11)), (18:1(12))-Pro(4-amino)-(18:1(12)), (18:1(3))-Pro(4-amino)-(18:1(5)), (18:1(3))-Pro(4-amino)-(18:1(7)), (18:1(3))-Pro(4-amino)-(18:1(9)), (18:1(3))-Pro(4-amino)-(18:1(11)), (18:1(3))-Pro(4-amino)-(18:1(12)), (18:1(5))-Pro(4-amino)-(18:1(7)), (18:1(5))-Pro(4-amino)-(18:1(9)), (18:1(5))-Pro(4-amino)-(18:1(11)), (18:1(5))-Pro(4-amino)-(18:1(12)), (18:1(7))-Pro(4-amino)-(18:1(9)), (18:1(7))-Pro(4-amino)-(18:1(11)), (18:1(7))-Pro(4-amino)-(18:1(12)), (18:1(9))-Pro(4-amino)-(18:1(11)), (18:1(9))-Pro(4-amino)-(18:1(12)), (18:1(11))-Pro(4-amino)-(18:1(12)), (18:1(3))-Pro(4-amino)-(18:2(9,12)), (18:1(5))-Pro(4-amino)-(18:2(9,12)), (18:1(7))-Pro(4-amino)-(18:2(9,12)), (18:1(9))-Pro(4-amino)-(18:2(9,12)), (18:1(11))-Pro(4-amino)-(18:2(9,12)), (18:1(12))-Pro(4-amino)-(18:2(9,12)), (18:2(9,12))-Pro(4-amino)-(18:1(3)), (18:2(9,12))-Pro(4-amino)-(18:1(5)), (18:2(9,12))-Pro(4-amino)-(18:1(7)), (18:2(9,12))-Pro(4-amino)-(18:1(9)), (18:2(9,12))-Pro(4-amino)-(18:1(11)), (18:2(9,12))-Pro(4-amino)-(18:1(12)), (18:2(9,12))-Pro(4-amino)-(18:2(9,12)), and a cationic form of any of the foregoing.

17. A composition comprising a compound of claim 1 contacted with an active agent.

18. A composition comprising a compound of claim 1 contacted with an active nucleic acid agent.

19. A composition comprising a compound of claim 1 contacted with an active RNA agent.

20. A composition comprising a compound of claim 1 contacted with a UsiRNA agent.

21. A composition comprising a compound of claim 1 contacted with a siRNA agent.

22. A composition comprising a compound of claim 1 admixed with a lipid, a cationic lipid, or a non-cationic lipid.

23. A method for delivering a therapeutic nucleic acid to a cell comprising contacting the cell with a formulation containing a compound according to claim 1 and a nucleic acid agent.

24. A method for inhibiting expression of a gene in a cell comprising contacting the cell with a formulation containing a compound according to claim 1 and a nucleic acid agent.

25. A method for inhibiting expression of a gene in a mammal comprising administering to the mammal a formulation containing a compound according to claim 1 and a nucleic acid agent.

26. A method for treating a disease in a human comprising administering a formulation containing a compound according to claim 1 and a nucleic acid agent to the human, wherein the disease is cancer, bladder cancer, cervical cancer, liver cancer, liver disease, hypercholesterolemia, an inflammatory disease, a metabolic disease, inflammation, arthritis, rheumatoid arthritis, encephalitis, bone fracture, heart disease, and viral disease.

27-29. (canceled)
